(19) United States
(12) Patent Application Publication
(10) Pub. No.: US 2023/0215576 A1
(43) Pub. Date: Jul. 6, 2023

(54) METHOD FOR ASSESSING HISTOLOGICAL DATA OF AN ORGAN AND ASSOCIATED DEVICES

(71) Applicants: INSTITUT NATIONAL DE LA SANTE ET DE LA RECHERCHE MEDICALE (INSERM), Paris (FR); ASSISTANCE PUBLIQUE-HOPITAUX DE PARIS, Paris (FR); UNIVERSITE PARIS CITÉ, Paris (FR)

(72) Inventors: Alexandre LOUPY, Paris (FR); Olivier AUBERT, Paris (FR); Xavier JOUVEN, Paris (FR)

(21) Appl. No.: 18/000,871

(22) PCT Filed: Jun. 8, 2021
(86) PCT No.: PCT/EP2021/065230

§ 371 Date: Dec. 6, 2022

(30) Foreign Application Priority Data
Jun. 10, 2020 (EP) 20305629.6

(51) Int. Cl. G16H 50/30; G16H 50/20; G16H 10/60; G16H 10/20

(52) U.S. Cl. CPC G16H 50/30; G16H 50/20; G16H 10/60; G16H 10/20

(57) ABSTRACT

The present invention relates to the field of artificial intelligence used in the medical context. The tissue biopsy is an invasive method that is widely used to obtain histological information that are useful notably for diagnosing. The inventors have thus searched to limit such invasive character. For this, it is proposed a method for predicting the result of a tissue biopsy, which provides accurate results without carrying out a biopsy.

### TECHNICAL FIELD OF THE INVENTION

The present invention concerns a method for assessing at least one histological piece of information of an organ of a subject. The present invention also relates to methods carrying out the steps of a method for assessing, the methods being selected among a method for predicting that a subject is at risk of suffering from a disease, a method for diagnosing a disease, a method for identifying a therapeutic target for preventing and/or treating a disease, a method for identifying a biomarker for a disease, a method for screening a compound useful as a medicament and a method for monitoring patients enrolled in a clinical trial. The present invention also concerns a computer program product and a computer-readable medium involved in these methods.

### BACKGROUND OF THE INVENTION

Organ transplantation is currently recognised as the treatment of choice for patients with end-stage renal disease (ESRD), which is an underestimated but increasing burden worldwide. Indeed, chronic kidney disease (CKD) affects 850 million individuals worldwide (in comparison, diabetes, cancer, and HIV/AIDS affect 422, 42, and 37 million individuals worldwide, respectively). Despite the progress made in immunosuppressive regimens, thousands of allografts are failing every year, with immediate consequences for the patients in terms of mortality, morbidity and cost for the society. Recently, it has been shown that the loss of a kidney allograft represents nowadays an important cause of ESRD.

To predict such issue, it is known to perform day-zero biopsies at the time of transplantation to obtain histological information. The histological evaluation of donors implemented in some transplant programs was used by clinicians to judge the quality of a donor organ and, on occasion, to rule out the possibility of underlying disease in donors.

Another advantage of the donor organ biopsy sample relies on the fact that it provides a valuable baseline against which, the results of subsequent biopsies of the renal allograft can be compared to and may also advocate therapeutic strategies.

Day-zero biopsy also serves to discriminate after transplantation the histological lesions that are donor transmitted or acquired after transplantation. Additionally, day-zero biopsies are used to optimize proper allocation process.

However, biopsy is an invasive, time-consuming and costly procedure and requires surgical, medical and pathological resources.

### SUMMARY OF THE INVENTION

There is therefore a need for a method for obtaining the histological pieces of information of an organ of a subject which limits the invasive character of a biopsy.

As a summary, the present invention relates to the field of artificial intelligence used in the medical context. The tissue biopsy is an invasive method that is widely used to obtain histological information that are useful notably for diagnosing. The inventors have thus searched to limit such invasive character.

Recently, this question has become even more challenging in the context of organ shortage. This is due to increase in transplantation of kidneys from older donors, from donation after cardiac death and from donors with significant clinical risk factors. These vulnerable organs may carry at the time of transplantation, lesions of arteriosclerosis, atrophy fibrosis, arterial hyalinosis and glomerulosclerosis, which might be wrongly attributed to drug toxicity, infectious diseases or allo-immune response if seen post transplantation, because of their non specificity

For this, it is proposed a method for predicting the result of a tissue biopsy, which provides accurate results without carrying out a biopsy.

To the end, the specification describes a method for assessing at least one histological piece of information of an organ of a subject, notably a graft from a donor, the method being computer-implemented and the method comprising providing parameters relative to the subject, and, for each of the at least one histological piece of information, applying a predicting function on the provided subject data to obtain an assessed histological piece of information, the assessed histological piece of information being a numerical value for the organ when the histological piece of information is a numerical value or the assessed histological piece of information being probabilities of belonging to different predefined classes for the organ when the histological piece of information is a belonging to a predefined class among the different predefined classes, and each predicting function being specific to the considered histological piece of information and being obtained by using an artificial intelligence technique.

With such method, the histological pieces of information are accessible in a fast and easy way.

Indeed, the method does not require any biopsy and thus no invasive or surgical acts.

In addition, for the care unit, there is only to enter data in a terminal carrying out the method for assessing. Only few resources are thus involved. In particular, no laboratory is involved in the method for assessing.

In other words, the present method for assessing provides clinicians with a virtual biopsy tool in order to guide diagnostics, therapeutics and immediate patient management post-transplant and to minimize additional post-operational issues.

According to further aspects of this method for assessing which are advantageous but not compulsory, the method for assessing might incorporate one or several of the following features, taken in any technically admissible combination:
for each of the at least one histological piece of information, the artificial intelligence technique comprises a phase of preparing a data set formed by elements, each element associating to subject parameters the assessed histological piece of information, a phase of training a plurality of models, to obtain trained models, and a phase of obtaining the predicting function comprising selecting models among the plurality of trained models based on a performance criteria, to obtain selected models, and obtaining the predicting function as a aggregating function of the selected models.
the organ is a kidney, the histological pieces of information being the value of the glomerusclerosis and the predefined class being the stages of the arteriosclerosis, the stages of the arteriolar hyalinosis and the stages of the interstitial fibrosis/tubular atrophy, the predefined class being preferably the class of the international Banff classification of allograft pathology.
the organ is a heart, the histological pieces of information being the stages of the acute cellular rejection, the stages of the antibody-mediated rejection the predefined class being preferably the class of the International Society for Heart and Lung Transplantation or international Banff classification of allograft pathology.
the organ is a lung, the histological pieces of information being the stages of the acute cellular rejection, the stages of the antibody-mediated rejection the predefined class being preferably the class of the International Society for Heart and Lung Transplantation or international Banff classification of allograft pathology.
the provided subject parameters comprise at least one piece of information chosen among the list consisting of the comorbidities, the comorbidities being, for instance, a binary response to a question chosen among the following questions: whether the donor is living or deceased, and if deceased, whether the cause of the death is due to a circulatory illness or due to a cerebrovascular cause, whether the donor suffers from hypertension, whether the donor suffers from diabetes, whether the donor suffers from Hepatitis C virus, a clinical data, the clinical data being, for instance, a data chosen among the age of the donor, the gender of the donor, the ethnicity of the donor and the body mass index of the donor, and a biological data, the biological data being, for instance, a data chosen among the proteinuria rate and the creatinine rate of the donor.
the phase of preparing a data set formed by elements comprises carrying out at least one preparation procedure, the preparation procedure being a preparation technique chosen among a first procedure comprising collecting initial elements, and completing the initial elements by using an imputation technique, the imputation technique comprising using a random forest technique, a second procedure comprising splitting the data set into a training set and a testing set, and a third procedure comprising the phase of preparing comprises a standardization of the subject parameters, notably by calculating the ratio of the difference between the subject parameter and the mean of the same subject parameters and the standard deviation of the same subject parameters.
when the histological piece of information is a belonging to a predefined class among different predefined classes and the different predefined classes being superior or equal to 4, the initial training data set comprises a respective number of elements for each predefined class of the considered histological piece of information, the phase of preparing comprising itering an operation of replacing randomly an element present in the training data set with a first number superior to at least one other numbers by elements present in the training data set with an inferior number to the first number until the number of elements for each predefined class be the same in the obtained training data set.
the phase of training comprises penalizing in case of mispredicting of the two uppest classes and/or, wherein each model comprises hyperparameter for controlling the training process and the phase of training comprising hyperparameter tuning.
the phase of training comprises creating heterogeneities in the set of data.
the creating of heterogeneities comprises using repeated k-fold cross validation or bootstrapping.
the models are chosen in the list consisting of a linear model, the linear model being, for instance, penalized multinomial regression or linear discriminant analysis, a non-linear model, the non-linear model being, for instance, a radial support vector machine, an ensemble model, the ensemble model being, for instance, chosen in the list consisting of random forests, gradient boosting machines, extreme gradient boosting tree and naïve Bayes, and a deep learning model, the deep learning model being, for instance, a neural network or a model averaged neural network.
the artificial technique comprises an evaluation phase, the evaluation phase comprises carrying out at least one evaluation procedure, the evaluation procedure being an evaluation procedure chosen among a first procedure comprising applying multi-AUC of unweighted pairwise discriminability of classes when the histological piece of information is a belonging to a predefined class among the different predefined classes, a second procedure comprising, for each histological piece of information which is a numerical value, calculating the mean absolute error between the predicted value and the measured value for the histological piece of information, a third technique comprising using a robustness test and/or a durability test, a fourth technique comprising a random forest algorithm, and a fifth technique comprising using a bootstrapping technique.
the aggregating function is chosen in the list consisting of: simple average, weighted average, majority voting, weighted voting and ensemble stacking.

The specification also describes a Method selected from the group consisting of:
a method for predicting that a subject is at risk of suffering from a disease, the method for predicting comprising at least the steps of:
   carrying out the steps of a method for assessing at least one histological piece of information as previously described wherein the step of providing is achieved by receiving the parameters relative to the subject at risk of suffering from a disease, to obtain assessed histological pieces of information, and
   predicting that the subject is at risk of suffering from the disease based on the assessed histological pieces of information,
a method for diagnosing a disease to a subject, the method for diagnosing comprising at least the steps of:
   carrying out the steps of the method for assessing at least one histological piece of information as previously described wherein the step of providing is achieved by receiving the parameters relative to the subject, to obtain assessed histological pieces of information, and
   diagnosing the disease based on the assessed histological pieces of information,
a method for identifying a therapeutic target for preventing and/or treating a disease, the method comprising at least the steps of:
   carrying out the steps of the method for assessing at least one histological piece of information of an organ of a first subject, to obtain first assessed histological pieces of information, wherein the first subject is suffering from the disease and the assessing method is as previously described wherein the step of providing is achieved by receiving the parameters relative to the subject,
   carrying out the steps of the method for assessing at least one histological piece of information of an organ of a second subject, to obtain second assessed histological pieces of information, wherein the second subject is not suffering from the disease and the assessing method is as previously described wherein the step of providing is achieved by receiving the parameters relative to the subject, and
   selecting a therapeutic target based on the comparison of the first and second assessed histological pieces of information,
a method for identifying a biomarker for a disease, the biomarker being a diagnosis biomarker of the disease, a susceptibility biomarker of the disease, a prognostic biomarker of the disease or a predictive biomarker in response to the treatment of the disease, the method comprising at least the steps of:
   carrying out the steps of the method for assessing at least one histological piece of information of an organ of a first subject, to obtain first assessed histological pieces of information, wherein the first subject is suffering from the disease and and the assessing method is as previously described wherein the step of providing is achieved by receiving the parameters relative to the subject,
   carrying out the steps of the method for assessing at least one histological piece of information of an organ of a second subject, to obtain second assessed histological pieces of information, wherein the second subject is not suffering from the disease and the assessing method is as previously described wherein the step of providing is achieved by receiving the parameters relative to the subject, and
   selecting a biomarker target based on the comparison of the first and second assessed histological pieces of information, and
a method for screening a compound useful as a medicament, the compound having an effect on a known therapeutical target for preventing and/or treating a disease, the method comprising at least the steps of:
   carrying out the steps of the method for assessing at least one histological piece of information of an organ of a first subject, to obtain first assessed histological pieces of information, wherein the first subject is from the disease and has received the compound and the assessing method is as previously described wherein the step of providing is achieved by receiving the parameters relative to the subject,
   carrying out the steps of the method for assessing at least one histological piece of information of an organ of a second subject, to obtain second assessed histological pieces of information, wherein the second subject is suffering from the disease and has not received the compound and the assessing method is as previously described wherein the step of providing is achieved by receiving the parameters relative to the subject, and
   selecting a biomarker target based on the comparison of the first and second assessed histological pieces of information, and
a method for monitoring patients enrolled in a clinical trial to provide a quantitative measure for the therapeutic efficacy of the therapy which is subject to the clinical trial by carrying out the steps of the method for assessing at least one histological piece of information of an organ of said patients, the assessing method being as previously described wherein the step of providing is achieved by receiving the parameters relative to the subject.

The specification also relates to a computer program product comprising computer program instructions, the computer program instructions being loadable into a data-processing unit and adapted to cause execution of a method as previously described when run by the data-processing unit.

The specification further describes a computer-readable medium comprising computer program instructions which, when executed by a data-processing unit, cause execution of a method as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 is a schematic view of a system adapted to carry out a method for assessing,

FIG. 2 is a functional view of an example of a method for assessing,

FIG. 3 is a flowchart illustrating the carrying out of a specific artificial intelligence technique which is used in the method for assessing of FIG. 2, and

FIG. 4 is a schematic view of a step of the example of the artificial intelligence technique illustrated in FIG. 3.

### DETAILED DESCRIPTION OF SOME EMBODIMENTS

Description of the System

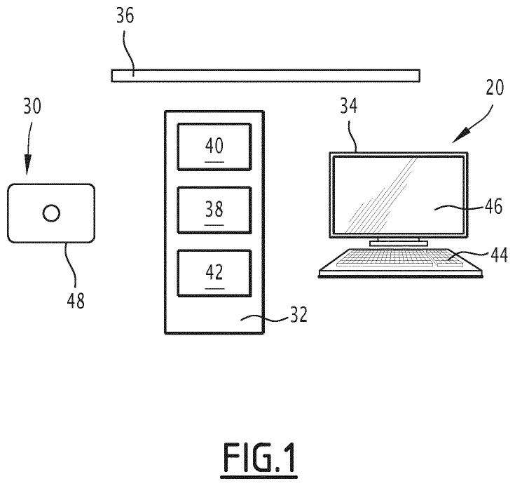

A system 20 and a computer program product 30 are represented on FIG. 1. The interaction between the computer program product 30 and the system 20 enables to carry out a method for assessing at least one histological piece of information of an organ of a subject as will be described later. Such method for assessing is named “assessing method” in the remainder of the specification.

This assessing method is a computer-implemented method.

The system 20 is a desktop computer. In variant, the system 20 is a rack-mounted computer, a laptop computer, a tablet computer, a PDA or a smartphone.

In specific embodiments, the system 20 is adapted to operate in real-time and/or is an embedded system, notably in a vehicle such as a plane.

In the case of FIG. 1, the system 20 comprises a calculator 32, a user interface 34 and a communication device 36.

The calculator 32 is electronic circuitry adapted to manipulate and/or transform data represented by electronic or physical quantities in registers of the calculator 32 and/or memories in other similar data corresponding to physical data in the memories of the registers or other kinds of displaying devices, transmitting devices or memoring devices.

As specific examples, the calculator 32 comprises a monocore or multicore processor (such as a CPU, a GPU, a microcontroller and a DSP), a programmable logic circuitry (such as an ASIC, a FPGA, a PLD and PLA), a state machine, gated logic and discrete hardware components.

The calculator 32 comprises a data-processing unit 38 which is adapted to process data, notably by carrying out calculations, memories 40 adapted to store data and a reader 42 adapted to read a computer-readable medium.

The user interface 34 comprises an input device 44 and an output device 46.

The input device 44 is a device enabling the user of the system 20 to input information or command to the system 20.

In FIG. 1, the input device 44 is a keyboard. Alternatively, the input device 44 is a pointing device (such as a mouse, a touch pad and a digitizing tablet), a voice-recognition device, an eye tracker or a haptic device (motion gestures analysis).

The output device 46 is a graphical user interface, which is a display unit adapted to provide information to the user of the system 20.

In FIG. 1, the output device 46 is a display screen for visual presentation of output. In other embodiments, the output device is a printer, an augmented and/or virtual display unit, a speaker or another sound generating device for audible presentation of output, a unit producing vibrations and/or odors or a unit adapted to produce electrical signal.

In a specific embodiment, the input device 44 and the output device 46 are the same component forming man-machine interfaces, such as an interactive screen.

The communication device 36 enables unidirectional or bidirectional communication between the components of the system 20. For instance, the communication device 36 is a bus communication system or an input/output interface.

The presence of the communication device 36 enables that, in some embodiments, the components of the system 20 be remote one from another.

The computer program product 30 comprises a computer-readable medium 48.

The computer-readable medium 48 is a tangible device that can be read by the reader 42 of the calculator 32.

Notably, the computer-readable medium 48 is not transitory signals per se, such as radio waves or other freely propagating electromagnetic waves, such as light pulses or electronic signals.

Such computer-readable storage medium 48 is, for instance, an electronic storage device, a magnetic storage device, an optical storage device, an electromagnetic storage device, a semiconductor storage device or any combination thereof.

As a non-exhaustive list of more specific examples, the computer-readable storage medium 48 is a mechanically encoded device such a punchcards or raised structures in a groove, a diskette, a hard disk, a ROM, a RAM, an EROM, an EEPROM, a magnetic-optical disk, a SRAM, a CD-ROM, a DVD, a memory stick, a floppy disk, a flash memory, a SSD or a PC card such as a PCMCIA.

A computer program is stored in the computer-readable storage medium 48. The computer program comprises one or more stored sequence of program instructions.

Such program instructions when run by the data-processing unit 38, cause the execution of steps of any method that will be described below.

For instance, the form of the program instructions is a source code form, a computer executable form or any intermediate forms between a source code and a computer executable form, such as the form resulting from the conversion of the source code via an interpreter, an assembler, a compiler, a linker or a locator. In variant, program instructions are a microcode, firmware instructions, state-setting data, configuration data for integrated circuitry (for instance VHDL) or an object code.

Program instructions are written in any combination of one or more languages, such as an object oriented programming language (FORTRAN, C″++, JAVA, HTML), procedural programming language (language C for instance).

Alternatively, the program instructions is downloaded from an external source through a network, as it is notably the case for applications. In such case, the computer program product comprises a computer-readable data carrier having stored thereon the program instructions or a data carrier signal having encoded thereon the program instructions.

In each case, the computer program product 30 comprises instructions, which are loadable into the data-processing unit 38 and adapted to cause execution of steps of any method described below when run by the data-processing unit 38. According to the embodiments, the execution is entirely or partially achieved either on the system 20, that is a single computer, or in a distributed system among several computers (notably via cloud computing).

Operating of the System

The operating of the system 20 is now described in reference to an example of carrying out an assessing method, that is a method for assessing several histological pieces of information of a kidney of a subject, the kidney being a graft from a donor.

The term “donor” refers to the subject that provides the organ and/or tissue transplant or graft to be transplanted into the recipient.

This means that, in such case which is presented in this paragraph, the term “subject” designates the recipient that receives an organ and/or tissue transplant or graft obtained from a donor.

In this specific example, the subject is a human being.

More generally, the subject is a living subject and notably an animal.

For instance, the subject is a mammal, and more specifically a rodent such a mouse.

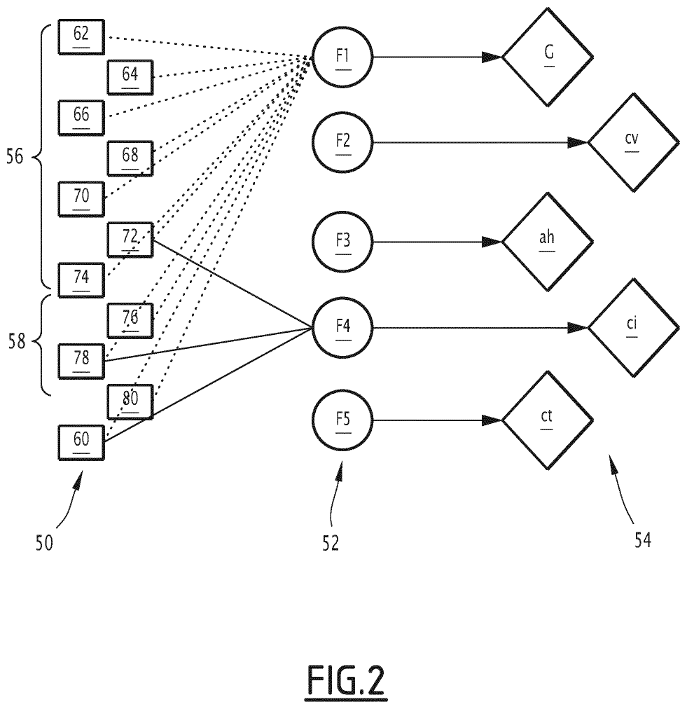

As represented schematically on FIG. 2, the assessing method is a method that associates to parameters 50 relative to the subject (named subject parameters 50) several histological pieces of information 54 via the application of one or several functions 52. In this FIG. 2, the subject parameters 50 are each represented by a rectangle, the histological pieces of information 54 are each represented by a diamond and the one of several functions 52 are each represented by a circle.

In other words, the assessing method comprises a providing step and an applying step.

At the step of providing, subject parameters 50 are provided.

For instance, a user enters data in the input device 44.

Alternatively, the parameters are received by the system 20, notably from a remote server.

In the present example, the provided subject parameters comprise the comorbidities 56, clinical data 58 and biological data 60.

By definition, a comorbidity is the presence of one or more additional conditions co-occurring with a primary condition.

In the present example, the comorbidities 56 are a binary response to several questions. The questions are the following:
whether the donor is living or deceased (item 62 in FIG. 2),
if the donor is deceased, whether the cause of the death is due to a circulatory illness, such as a cardiac illness (item 64 in FIG. 2),
if the donor is decease, whether the cause of the death is due to a cerebrovascular cause (item 66 in FIG. 2),
whether the donor suffers from hypertension (item 68 in FIG. 2),
whether the donor suffers from diabetes (item 70 in FIG. 2),
whether the donor suffers from proteinuria (item 72 in FIG. 2), and
whether the donor suffers from Hepatitis C virus (item 74 in FIG. 2).

In variant or in complement, the provided subject parameters 50 comprises the comorbidities of the subject, notably chosen among the previous list of items 62 to 74.

The term “proteinuria” refers to a condition in which excess protein is present in the urine of a subject. In human subjects, proteinuria is often diagnosed by urinalysis. Clinically, proteinuria is expressed as a ratio for urinary protein/creatinine (g/g of creatinine) and said ratio is normally comprised between 0 and 0.3. When such ratio is out of this range, it is considered that the subject suffers from proteinuria.

In the described example, the clinical data 58 is:
the age of the donor (item 76 in FIG. 2),
the gender of the donor (item 78 in FIG. 2), and
the body mass index of the donor (item 80 in FIG. 2). The body mass index is the donor's weight in kilograms divided by the square of height in meters.

As an illustration, the biological data 60 is the creatinine rate in milligrams (mg) per deciliter (dL). Normal levels of creatinine in the blood are approximately 0.7 to 1.2 milligram (mg) per deciliter (dL) in adult males and 0.5 to 1.0 milligram per deciliter in adult females.

By definition, a histological piece of information is a data/information which concerns the study of biological tissues.

In the present example, the histological pieces of information 54 are allograft histological pieces of information.

In this specific example, the histological pieces of information 54 are assessed according to the Banff Classification that is an international consensus classification for the reporting of biopsies from solid organ transplants. Banff Lesion Scores indeed assess the presence and the degree of histopathological changes in the different compartments of renal transplant biopsies, focusing primarily but not exclusively on the diagnostic features seen in rejection.

In the current illustrated case, the histological pieces of information 52 are four pieces of information which are:
the value of the percentage of the glomerusclerosis,
the stage of arteriosclerosis,
the stage of the arteriolar hyalinosis,
the stage of interstitial fibrosis, and
the stage of tubular atrophy.

In this case, the stages are the class of the Banff classification of Renal Allograft Pathology predefined classes.

More specifically, glomerulosclerosis is hardening of the glomeruli in the kidney. It is a general term to describe scarring of the kidneys' tiny blood vessels, the glomeruli, the functional units in the kidney that filter urea from the blood. By definition, the value of the glomerusclerosis is a percentage obtained by dividing the number of sclerosed glomeruli by the number of glomeruli found on the biopsy. Such value is represented by the item G on FIG. 2.

The arteriosclerosis is evaluated by Banff Lesion Score cv. This score reflects the extent of arterial intimal thickening in the most severely affected artery. The score is evaluated according to four stages which are:
cv0: no chronic vascular changes;
cv1: vascular narrowing of up to 25% luminal area by fibrointimal thickening;
cv2: vascular narrowing of 26 to 50% luminal area by fibrointimal thickening, and
cv3: vascular narrowing of more than 50% luminal area y fibrointimal thickening.

The arteriolar hyalinosis is evaluated by Banff Lesion Score ah. This score evaluates the extent of arteriolar hyalinosis. The score is evaluated according to four stages which are:
stage 0: no PAS (PAS)-positive hyaline arteriolar thickening;
stage 1: mild to moderate PAS-positive hyaline thickening in at least 1 arteriole;
stage 2: moderate to severe PAS-positive hyaline thickening in more than 1 arteriole, and
stage 3: severe PAS-positive hyaline thickening in many arterioles.

The interstitial fibrosis is evaluated by Banff Lesion Score ci. This score evaluates the extent of cortical fibrosis. The score is evaluated according to four stages which are:
ci0: interstitial fibrosis in up to 5% of cortical area;
ci1: interstitial fibrosis in 6 to 25% of cortical area (mild interstitial fibrosis);
ci2: interstitial fibrosis in 26 to 50% of cortical area (moderate interstitial fibrosis), and
ci3: interstitial fibrosis in more than 50% of cortical area (severe interstitial fibrosis).

The interstitial fibrosis is evaluated by Banff Lesion Score ct. This score evaluates the extent of cortical tubular atrophy which is usually tightly associated with the areas affected with interstitial fibrosis. The score is evaluated according to four stages which are:
ct0: no tubular atrophy;
ct1: tubular atrophy involving up to 25% of the area of cortical tubules;
ct2: tubular atrophy involving 26 to 50% of the area of cortical tubules, and
ct3: tubular atrophy involving more than 50% of the area of cortical tubules.

As apparent on FIG. 2, there is one function for each histological piece of information 54. This means that each function is specific to one histological piece of information 54.

In other words, in the current case, there are five functions named F1, F2, F3, F4 and F5. The first function F1 predicts the value of the glomerusclerosis (G), the second function F2 predicts the stage of arteriosclerosis (cv), the third function F3 predicts the stage of the arteriolar hyalinosis (ah), the fourth function F4 predicts the stage of interstitial fibrosis (ci) and the fifth function F5 predicts the stage of tubular atrophy (ct).

Each predicting function F1, F2, F3, F4 or F5 associates to subject parameters 50 provided as inputs an output which is the histological piece of information 54 that the function F1, F2, F3, F4 or F5 is adapted to predict. The histological piece of information 54 which is thus predicted is at least one of assessed histological pieces of information 54 obtained by the assessing method.

As apparent from FIG. 2, each predicting function F1, F2, F3, F4 or F5 is applied on part or each of the subject parameters 50.

As an illustration, the first predicting function F1 is applied on each subject parameter 50 (see dotted lines) whereas the fourth predicting function F4 is only applied to three subject parameters 60, 72 and 80 (see solid lines).

Each predicting function F1, F2, F3, F4 or F5 is obtained by using an artificial intelligence technique.

An artificial intelligence technique consists in establishing a model (also named algorithm) based on data.

In particular, the artificial intelligence technique often implies learning the model. The term “machine learning” is thus employed to designate the fact that the model is learned by the machine based on data.

According to the case, the machine learning technique implies using a learning among a supervised learning, an unsupervised learning, a semi-supervised learning, a reinforcement learning, a self learning, a feature learning, a sparse dictionary learning, an anomaly detection learning, a robot learning and association rules learning.

In particular, in the present example, the machine learning technique is a supervised learning technique, a semi-supervised learning technique or a reinforcement learning technique.

The model used in the artificial intelligence technique can be chosen from various models/algorithms, such as computational models and algorithms for classification, clustering, regression and dimensionality reduction, such as neural networks, genetic algorithms, support vector machines, k-means, kernel regression and discriminant analysis.

More generally, the artificial intelligence technique may imply the use of one or several of the following elements: sums, ratios, and regression operators, such as coefficients or exponents, biomarker value transformations and normalizations (including, without limitation, those normalization schemes based on clinical parameters, such as clinical data 58, gender, age or ethnicity), rules and guidelines, statistical classification models, and neural networks, structural and syntactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others.

Alternatively or in complement, the artificial intelligence technique may imply the use of one or several of the following elements: Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), boosting, regression analysis, Information fuzzy networks (IFN), statistical classification, AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

Alternatively or in complement, the artificial intelligence technique may imply the use of one or several of the following elements: artificial neural network, Data clustering, Expectation-maximization algorithm, Self-organizing map, Radial basis function network, Vector Quantization, Generative topographic map, Information bottleneck method, and IBSEAD, rule learning algorithms such as Apriori algorithm, Eclat algorithm and FP-growth algorithm, hierarchical clustering, such as Single-linkage clustering and Conceptual clustering, partitional clustering such as K-means algorithm and Fuzzy clustering.

Alternatively or in complement, the artificial intelligence technique uses a reinforcement learning algorithm. Examples of reinforcement learning algorithms include, but are not limited to, temporal difference learning, Q-learning and Learning Automata.

Alternatively or in complement, the artificial intelligence technique uses Data Pre-processing.

More specifically, the model is chosen among a linear model, a non-linear model, an ensemble model and a deep learning model.

A linear model is a model that uses linear relation(s) between the inputs and the outputs.

In the present case, the linear model is penalized multinomial regression or linear discriminant analysis

A non-linear model is a model that uses non-linear relation(s) between the inputs and the outputs.

For the case of FIG. 2, the linear model is a radial support vector machine.

A radial support vector machine is a classifier enabling to search a high-dimensional decision boundary to separate classes and maximize the margin.

An ensemble model is a model that aggregates multiple models to reduce loss.

In the present case, the ensemble model is an aggregation of several models and notably an aggregation of random forests (such algorithms aggregates concurrent multiple trees to reduce loss), gradient boosting machines (such algorithm corresponds to sequential and additive decision trees to reduce loss by using gradients in the loss function), extreme gradient boosting tree (this is an algorithm more efficient, flexible, and regularized than gradient boosting), naïve Bayes (it is a very simple and efficient probabilistic classifier. Naïve Bayes naively (strongly) assumes all features are independent).

Deep learning model is a model that uses multiple layers to progressively extract higher level features from the raw input.

For the case of FIG. 2, the deep learning model is a model averaged neural network.

Like random forest, model averaged neural network creates multiple neural networks to average them into one.

The assessing method of FIG. 2 therefore enables to assess several histological pieces of information.

The assessing method is fast in so far as the only requirement is to provide the subject parameters 50.

In addition, such providing can be achieved easily and notably by using the medical file wrapper which is stored in the database of the medical center wherein the assessing method is used.

Furthermore, the assessing method enables avoiding all the drawbacks of carrying out a biopsy since none is carried out.

It is to be noted that the assessing method, by using an artificial intelligence technique, enables to obtain reliable and accurate prediction (in other words, assessment) of the histological pieces of information.

To improve such accuracy, one may consider implementing a specific artificial intelligence technique which is described in the following section.

Description of a Specific Artificial Intelligence Technique

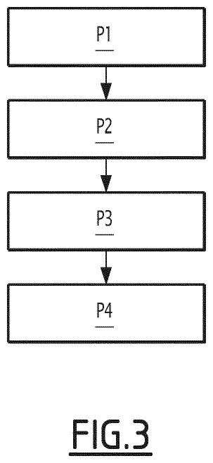

FIG. 3 is a flowchart illustrating a carrying out of the specific artificial intelligence technique for one function among the functions F1, F2, F3, F4 and F5.

As a specific example, FIG. 3 deals with the example of function F2 corresponding to the assessment of the stage of arteriosclerosis, this example being easily adapted to the other functions F1, F2, F3 or F5.

Such method is carried out by a system similar to the system 20.

In the case of FIG. 3, the artificial intelligence technique comprises fourth phases which are a phase of preparing P1, a phase of training P2, a phase of obtaining P3 and a phase of evaluating P4.

During the phase of preparing P1, a data set is formed.

The data set comprises several elements wherein each element associates to subject parameters 50 the assessed histological piece of information 54. In other words, the data set is a collection of data giving for many subject (for instance more than 100, preferably more than 1000) specific subject parameters 50 and the stage of arteriosclerosis.

In the specific example described, the phase of preparing P1 comprises an imputation step, a splitting step, an up-sampling step and a standardization step.

During the imputation step, initial elements are collected.

The initial elements are then completed by using an imputation technique.

In statistics, imputation is the process of replacing missing data with substituted values.

The imputation comprises using a random forest technique to select the elements that will be used for the missing data.

During the splitting step, the data set obtained at the end of the imputation step is split into a training set and a testing set.

For instance, ¾ of the elements of the data set obtained at the end of the imputation step are considered as the initial training set, the other elements being considered as the testing set.

Alternatively, ratios of 70/30 or 80/20 can be used at the splitting step.

During the up-sampling step, the initial training set is up-sampled.

This means that the initial training set is modified so that the number of elements for each stage of the training set be the same.

In practice, the initial training set comprises less and less elements as the stage becomes high because the higher stage is the less probable.

For the illustrated case, assuming that the initial repartition of the initial training set is 2000 elements for stage 0, 2000 elements for stage 1, 1000 elements for stage 2 and 300 elements for stage 3, the aim of the up-sampling step is to obtain a modified training set wherein the highest number of elements among the stages (here 2000) is the same for each stage. A modified training set is thus obtained with 8000 elements with 2000 elements for each stage.

For this, the up-sampling step comprises increasing the number of elements and iterating an operation of replacing.

The number of elements of the data set is increased by selecting randomly an element from the initial data set until the number of elements of the data set be equal to the number of stages (here 4) multiplied by the number of elements of the highest number of class (here 2000).

Then, an operation of replacing is iterated.

The operation of replacing consists in replacing randomly an element present in the training data set with a first number superior to at least one other numbers by elements present in the training data set with an inferior number to the first number.

For instance, here an element of the stage 1 is replaced by an element of stage 3 which is randomly chosen.

The operation of replacing is iterated until the number of elements for each stage is the same in the obtained training data set.

Alternatively, the up-sampling step is carried out by adding elements randomly chosen in the stage which are underrepresented in the initial training data set.

During the standardization step, at least some of the subject parameters 50 are standardized.

In other words, the standardization step is carried out so that quantitative subject parameters 50 are in a similar range which enables to foster the phase of training P2.

Examples of quantitative parameters are the age, the creatinine rate or the body mass index.

For instance, the value of the subject parameter 50 to standardize is replaced by the ratio of the difference between the current value of the subject parameter 50 and the mean of the value of said subject parameter 50 in the data set and the standard deviation of the said subject parameter 50 in the data set.

In the current case, the standardization step is applied both on the training data set and on the test data set but, alternatively, can be applied only on the training data set.

Alternatively, the phase of preparing P1 comprises carrying out not all the previously cited steps which all correspond to a preparing procedure.

In other embodiments, the steps of the phase of preparing P1 are carried out in a different order, for instance the standardization step is the first step which is carried out.

In any case, at the end of the phase of preparing P1, an appropriate training data set and an appropriate test data set are obtained.

During the phase of training P2, a plurality of models are trained.

The phase of training P2 is an unsupervised training.

In the present example, the phase of training comprises a training step, a creating step and a tuning step.

During the training step, the models are trained based on the appropriate training data set and an appropriate test data set.

Such training step is carried by penalizing in case of mispredicting of the two uppest stages of arteriosclerosis.

This means that the error function used to train the model is considering that an error of prediction when the prediction should have been stages 2 or 3 is more serious than an error of prediction when the prediction should have been stages 0 or 1.

This enables to obtain trained models that, in the case, have to be improved, here by carrying out the creating step and the tuning step.

In other embodiments, the trained models obtained at this step are the trained models obtained at the end of the phase of training P2.

During the creating step, heterogeneities are created in the set of data For instance, in the current example, a k-fold cross-validation is repeated.

For instance, 10-folds cross-validation are randomly repeated three times with a new training process during which hyperparameters of the model are tuned.

Such creating step enables to minimize chance of overfitting and possible sampling bias.

Alternatively or in complement, the creating step comprises using bootstrapping.

During the tuning step, hyperparameters of the model adapted for controlling the training process are tuned.

Such procedure which is also named hyperparameter optimization finds a tuple of hyperparameters that yields an optimal model which minimizes a predefined loss function on given independent data. The objective function takes a tuple of hyperparameters and returns the associated loss.

In the present case, the hyperparameter tuning is achieved by using the data obtained at the end of the creating step.

At the end of the phase of training P2, a plurality of trained models is obtained.

During the phase of obtaining P3, the predicting function F2 is obtained.

The phase of obtaining P3 comprises a selecting step and an obtaining step.

During the selecting step, several models among the plurality of trained models are selected based on a performance criteria.

The performance criteria is a metrics that enables to evaluate numerically the distance between the predicted histological piece of information and the true histological piece of information.

For numerical value, the mean absolute error between the predicted value and the measured value for the histological piece of information is calculated.

This is notably the case for the percentage of sclerotic glomeruli (see item G on FIG. 2).

For the estimation of stages, it can be used a multi-class AUC of unweighted pairwise discriminability of classes by Hand and Till's formula, which is:

$$A U {C}_{t o t a l} = \frac{2}{C ( C - 1 )} {\sum }_{\{ {c}_{i} , {c}_{j} \} ∈ C} A U C ( {c}_{i} , {c}_{j} )$$

where:
AUCₜₒₜₐₗ is the value of the metrics used for evaluating the model,
C is the number of classes (4 for the case of function F2),
AUC (cᵢ,cⱼ) is the area under the two-class ROC curve involving classes cᵢ and cⱼ. A receiver operating characteristic curve, or ROC curve, is a graphical plot that illustrates the diagnostic ability of a binary classifier system as its discrimination threshold is varied.

In the illustrated case, the performance criteria is fulfilled when the AUCₜₒₜₐₗ is above a predetermined threshold.

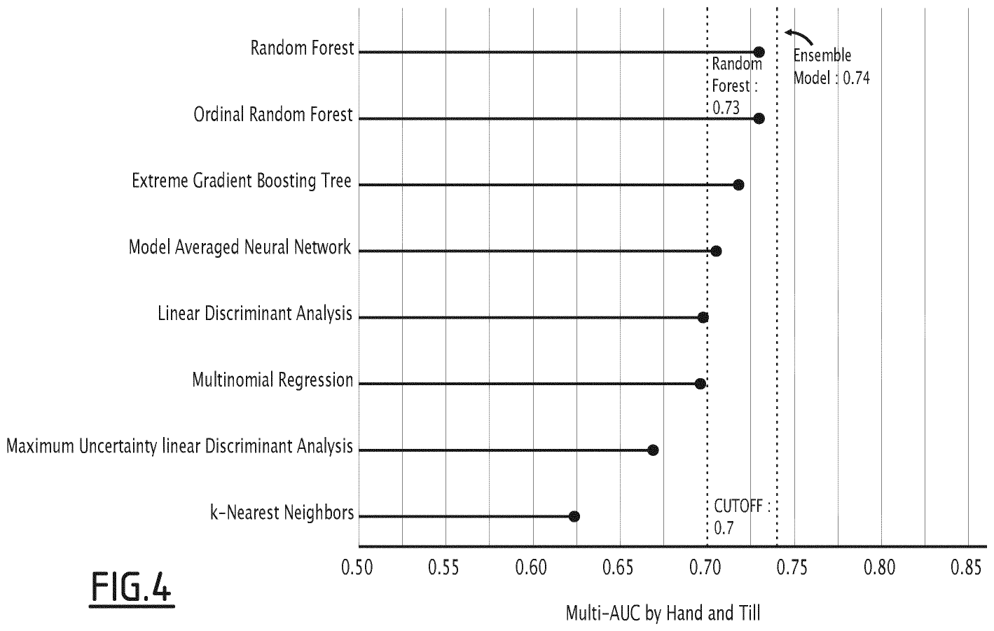

The results of a specific example of application is represented in FIG. 4.

The predetermined threshold is fixed to a cut-off value of 0.70.

The performance of eight models are given on FIG. 4.

The first model M1 is random forest, the second model M2 is ordinal random forest, the third model M3 is extreme gradient boosting tree, the fourth model M4 is model averaged network, the fifth model M5 is linear discriminant analysis, the sixth model M6 is multinomial regression, the seventh model M7 is maximum uncertainty linear discriminant analysis and the eightieth model M8 is k-nearest neighbors.

It appears that only the five first models M1 to M5 provide with performance above the predetermined threshold of 0.70.

Thus, in this specific example, the selected models are the five first models M1 to M5.

At the end of the selecting step, several selected models are thus obtained.

During the obtaining step, the predicting function F2 is obtained.

The predicting function F2 is an aggregating function of the selected models.

In other words, the predicting function F2 is a metaclassifier of the selected models or the predicting function F2 can be construed as a combination of multiple models into one super learner.

The aggregating function is, for instance, a majority voting.

In the present example, the predicting function F2 is thus a majority voting of the first five models which are models M1 to M5.

According to variants, the aggregating function is simple average, weighted average, a weighted voting or an ensemble stacking.

In such context, the ensemble stacking corresponds to applying a distinct function for the multiclass categorical variable(s) and the numeric variable(s), notably a simple averaging the probabilistic classifiers' results using the arithmetic mean (multiclass categorical variables) and a linear regression on the predicted results to minimize mean absolute error (numeric variable).

At the end of the obtaining step, the predicting function F2 is obtained.

During the phase of evaluating P4, the predicted function F2, and more specifically its performance, is evaluated.

The phase of evaluating P4 can be carried out by using the previous performance criteria.

This is the case in FIG. 4 wherein the total AUC of the predicting function F2 is equal to 0.74.

It can be noted that the performance of the predicting function F2 is better than the best performance of each model.

This is not surprising in so far as the predicting function F2 is taking the best of each model. Notably, when one model is wrong, if the other models are right, then the prediction of the predicting function F2 is right, resulting in a better performance.

Alternatively or in complement, the phase of evaluating P4 comprises using a robustness test and/or a durability test.

For instance, artificially created sequential errors on the test data sets are used to assess how performances of the models are sequentially reduced.

According to another variant or in complement, the phase of evaluating P4 comprises using a random forest algorithm. Such algorithm is used to examine the feature importance to predict the histological information.

Alternatively or in complement, the phase of evaluating P4 comprises using a bootstrapping technique. Such bootstrapping technique is used to generate confidence intervals on the prediction.

Other Embodiments of the Assessing Method

The present assessing method can be implemented in many different ways. Some examples are given below.

The assessing method may comprise additional step such as outputting the predicted histological piece of information.

The output can be a radar plot, an enumeration of values or so on.

Preferably, the output is displayed on the output device 46 of the system 20.

For instance, at the providing step, only specific subject parameters 50 are provided, for instance one or two among the comorbidities 56, the clinical data 58 or the biological data 60.

As another example, more subject parameters 50 are provided such as the ethnicity of the subject or the glomerular filtration rate.

The term “glomerular filtration rate” or “GFR” refers to the volume of fluid filtered from the renal (kidney) glomerular capillaries into the Bowman's capsule per unit time. GFR is used to assess renal function in a subject.

Such glomerular filtration rate is, for instance, the estimated GFR. The term “estimated GFR” or “eGFR” refers to an estimate of the Glomerular Filtration Rate or GFR, calculated using the Modification of Diet in Renal Disease (MDRD) equation developed by the Modification of Diet in Renal Disease Study Group described in Levey A S, Bosch J P, Lewis J B, Greene T, Rogers N, Roth D, “A more accurate method to estimate glomerular filtration rate from serum creatinine: a new prediction equation. Modification of Diet in Renal Disease Study Group” Ann. Intern. Med. 130 (6): 461-70 (1999), the contents of which are herein incorporation by reference. Typically, the unit of measurement for eGFR is mL/min/1.73 m². Typically, the eGFR is comprised between 0 and 120 mL/min/1.73 m².

The assessed histological piece(s) of information may be one or several of the histological pieces of information previously presented.

Preferably, one histological piece of information comprises the stage of at least three histological lesions.

The assessed histological piece(s) of information may also be evaluated differently.

For instance, instead of evaluating the ci and ct value, it can be considered to evaluate the interstitial fibrosis/tubular atrophy.

The interstitial fibrosis/tubular atrophy (IFTA) is evaluated by Banff Lesion Score i-IFTA. This score evaluates the extent of inflammation in scarred cortex. The score is assessed by different stages as follows:
stage 0 (i-IFTA0): No inflammation or less than 10% of scarred cortical parenchyma;
stage 1 (i-IFTA1): Inflammation in 10% to 25% of scarred cortical parenchyma;
stage 2 (i-IFTA2): Inflammation in 26% to 50% of scarred cortical parenchyma, and
stage 3 (i-IFTA3): Inflammation in more than 50% of scarred cortical parenchyma.

One can also consider evaluating a score not according to stages but also numerically.

For IFTA, one can output the percentage of inflammation without determining the stage.

Other histological pieces of information may be assessed such as microcirculation inflammation, interstitial inflammation and tubulitis and transplant glomerulopathy.

Microcirculation inflammation (corresponding to the combination glomerulitis and peritubular capillaritis) results from the addition of Banff Lesion Score g (score for glomerulitis)+Banff Lesion Score ptc (score for peritubular capillaritis).

Banff Lesion Score g evaluates the degree of inflammation within glomeruli. Glomerulitis is a form of microvascular inflammation and is a feature of activity and antibody interaction with tissue in antibody-mediated rejection. The score is assessed as follows:
g0: no glomerulitis;
g1: segmental or global glomerulitis in less than 25% of glomeruli;
g2: segmental or global glomerulitis in 25% to 75% of glomeruli, and
g3: segmental or global glomerulitis in more than 75% of glomeruli.

Banff Lesion Score ptc evaluates the degree of inflammation within peritubular capillaries (PTCs). Together with glomerulitis, peritubular capillaritis constitutes microvascular inflammation as a feature of active antibody-mediated rejection or chronic active antibody-mediated rejection. The score is assessed as follows:
ptc0: maximum number of leukocytes <3;
ptc1: at least 1 leukocyte cell in ≥10% of cortical PTCs with 3-4 leukocytes in most severely involved PTC;
ptc2: at least 1 leukocyte in ≥10% of cortical PTC with 5-10 leukocytes in most severely involved PTC, and
ptc3: at least 1 leukocyte in ≥10% of cortical PTC with >10 leukocytes in most severely involved PTC.

The interstitial inflammation and tubulitis results from the addition of Banff Lesion Score i (score for interstitial inflammation)+Banff Lesion Score t (score for tubulitis).

Banff Lesion Score i evaluates the degree of inflammation in nonscarred areas of cortex (“interstitial Inflammation”), which is often a marker of acute T cell-mediated rejection. The score is assessed as follows:
i0: no inflammation or in less than 10% of unscarred cortical parenchyma;
i1: inflammation in 10 to 25% of unscarred cortical parenchyma;
i2: inflammation in 26 to 50% of unscarred cortical parenchyma, and
i3: inflammation in more than 50% of unscarred cortical parenchyma.

Banff Lesion Score t evaluates the degree of inflammation within the epithelium of the cortical tubules (“tubulitis”). The presence of mononuclear cells in the basolateral aspect of the renal tubule epithelium is one of the defining lesion of acute T cell-mediated rejection in kidney transplants. The score is assessed as follows:
t0: no mononuclear cells in tubules or single focus of tubulitis only;
t1: foci with 1 to 4 mononuclear cells/tubular cross section (or 10 tubular cells);
t2: foci with 5 to 10 mononuclear cells/tubular cross section (or 10 tubular cells), and
t3: foci with >10 mononuclear cells/tubular cross section or the presence of areas of tubular basement membrane destruction accompanied by i2/i3 inflammation and t2 elsewhere.

The transplant glomerulopathy (cg) is evaluated by Banff cg Score. The score is based on the presence and extent of glomerular basement membrane (GBM) double contours or multilamination in the most severely affected glomerulus. The score is assessed as follows:
cg0: no GBM double contours by light microscopy (LM) or electronic microscopy (EM);
cg1a: no GBM double contours by LM but GBM double contours (incomplete or circumferential) in at least 3 glomerular capillaries by EM, with associated endothelial swelling and/or subendothelial electron-lucent widening;
cg1b: double contours of the GBM in 1-25% of capillary loops in the most affected nonsclerotic glomerulus by LM; EM confirmation is recommended if EM is available;
cg2: double contours affecting 26 to 50% of peripheral capillary loops in the most affected—glomerulus, and
cg3: Double contours affecting more than 50% of peripheral capillary loops in the most affected-glomerulus.

This is all the more true when considering the fact that assessing histological pieces of information 54 is not only made for biopsy in the context of transplantations. Biopsy is also used for other context, such as kidney disease diagnosis or kidney cancer.

Other histological pieces of information 54 are also involved since such assessment method can be advantageously used in other medical acts such as smear, puncture liquid or kidney resection.

In addition, the assessment method is carried out on another organ.

For instance, instead of considering a graft which is a kidney, the graft is a heart or a lung or a liver.

In such case, one may consider other histological pieces of information 54 according to the organ considered.

Notably, if the organ is a heart, the histological pieces of information 54 are stages of the acute cellular rejection, the stages of the antibody-mediated rejection the predefined class being preferably the class of the International Society for Heart and Lung Transplantation or international Banff classification of allograft pathology.

As another example, if the a lung, the histological pieces of information 54 are stages of the acute cellular rejection, the stages of the antibody-mediated rejection the predefined class being preferably the class of the International Society for Heart and Lung Transplantation or international Banff classification of allograft pathology.

In addition, due to the fact that such assessment method can be used on other organs, such assessment method can be advantageously used in other medical acts such as endomyocardial or transbronchial or lung or liver biopsy, smear, puncture liquid and also lung or liver resection.

Each of the previous embodiments share the common features according to which the assessing method is a method for assessing at least one histological piece of information 54 of an organ of a subject, notably a graft from a donor, the method being computer-implemented and the method comprising providing parameters relative to the subject 50, and, for each of the at least one histological piece of information, applying a predicting function 52 on the provided subject data to obtain an assessed histological piece of information. The assessed histological piece of information 54 is a numerical value for the organ when the histological piece of information 54 is a numerical value or the assessed histological piece of information 54 being probabilities of belonging to different predefined classes for the organ when the histological piece of information 54 is a belonging to a predefined class among the different predefined classes. In addition, each predicting function 52 is specific to the considered histological piece of information and is obtained by using an artificial intelligence technique.

Such assessing method enables, in each case, to obtain accurate histological piece(s) of information with a non-invasive technique.

Such method is, in addition, easy to implement since such method can be carried out by entering subject parameters 50 which are generally known or that can be measured in a non-invasive way. Such entering action as well as carrying out the method can be achieved by using a system 20 which is generally available in each care unit.

In case the system 20 does not have the necessary calculation capabilities for applying the predicting function, calculation can be carried out by interacting with a remote server.

In each of these cases, there is no need of additional hardware resource in the care unit.

Besides, as no invasive act is carried out, the resource allocated to carry out the invasive acts is saved and can be allocated to other tasks.

This means that the assessing method is saving resources of the care unit while providing with the same information than the invasive act, such as a biopsy.

Applications

Such advantages of the assessing method renders this method appropriate for many applications linked to diseases.

According to the context and in accordance with the previously mentioned examples, such disease can be a kidney disease or a heart disease.

Other examples of diseases are acute cellular rejection, antibody mediated rejection, recurrence of the original disease (amyloidosis, diabetes notably) and poliomavirus nephropathy.

Graft loss, graft rejection, graft versus host disease, stenosis, thrombosis, acute tubulonephritis, chronic transplant nephropathy, kidney failure, atherosclerosis, arterial hypertension, coronary artery disease are other examples of such kind of diseases.

In each application, there exists a link between the disease of the application in which the assessing method is used and the organ to which the assessing method is applied. In other words, such disease is a disease or a disorder related to this organ.

It can thus be considered to use the assessing method in a method for predicting that a subject is at risk of suffering from a disease.

The term “risk” relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event).

The method for predicting comprises at least the steps of carrying out the steps of the assessing method on the subject, to obtain assessed histological pieces of information, and predicting that the subject is at risk of suffering from the disease based on the assessed histological pieces of information.

Alternatively, it can be considered a method for diagnosing a disease wherein the method for diagnosing comprises at least the steps of carrying out the steps of the assessing method, to obtain assessed histological pieces of information, and diagnosing the disease based on the assessed histological pieces of information.

The assessing method can also be advantageously used in a method for identifying a therapeutic target for preventing and/or treating a disease, the method comprising at least the steps of carrying out the steps of the method for assessing at least one histological piece of information of an organ of a first subject, to obtain first assessed histological pieces of information, the first subject being a subject suffering from the disease, carrying out the steps of the method for assessing at least one histological piece of information of an organ of a second subject, to obtain second assessed histological pieces of information, the second subject being a subject not suffering from the disease, and selecting a therapeutic target based on the comparison of the first and second assessed histological pieces of information.

Alternatively, it can be considered a method for identifying a biomarker for a disease, the biomarker being a diagnosis biomarker of the disease, a susceptibility biomarker of the disease, a prognostic biomarker of the disease or a predictive biomarker in response to the treatment of the disease, the method comprising at least the steps of carrying out the steps of the method for assessing at least one histological piece of information of an organ of a first subject, to obtain first assessed histological pieces of information, the first subject being a subject suffering from the disease, carrying out the steps of the method for assessing at least one histological piece of information of an organ of a second subject, to obtain second assessed histological pieces of information, the second subject being a subject not suffering from disease, and selecting a biomarker target based on the comparison of the first and second assessed histological pieces of information.

The assessing method can also be advantageously used in a method for screening a compound useful as a medicament, the compound having an effect on a known therapeutical target for preventing and/or treating a disease, the method comprising at least the steps of carrying out the steps of the method for assessing at least one histological piece of information of an organ of a first subject, to obtain first assessed histological pieces of information, the first subject being a subject suffering from the disease and having received the compound, carrying out the steps of the method for assessing at least one histological piece of information of an organ of a second subject, to obtain second assessed histological pieces of information, the second subject being a subject suffering from the disease and not having received the compound, and selecting a biomarker target based on the comparison of the first and second assessed histological pieces of information.

The assessing method is also advantageous in a method for monitoring patients enrolled in a clinical trial to provide a quantitative measure for the therapeutic efficacy of the therapy which is subject to the clinical trial by carrying out the steps of the assessing method on said patients.

More generally, the assessing method can advantageously be used in any context where the histological piece of information is used and, even more in the case where such histological piece of information can only be obtained in an invasive way.

In addition, the person skilled in the art can consider any combination of the features of the previously mentioned embodiment of the assessing method to obtain new embodiments when the features are technically compatible.

EXPERIMENTAL SECTION

This section is devoted to a study carried out by the Applicant, which shows the advantages of the present invention.

RESEARCH IN CONTEXT

Evidence Before this Study

In medicine, tissue biopsies are routinely performed to determine diagnosis, guide therapeutics and prognosis assessment. In kidney transplantation, day-zero biopsies are used as baseline status of the kidney allograft to better contextualize lesions found on subsequent allograft biopsies and guide decision making process. However, biopsy remains an invasive and costly procedure that mobilizes human resources, thereby delaying the transplantation procedure.

The Applicant has searched PubMed and MEDLINE from January 2000 to January 2021, using the terms (“noninvasive” or “non-invasive”), “biopsy”, “predict”, and “machine learning”, without language restrictions. This search found 164 studies. After removing 12 studies predicting a single disease diagnosis (e.g. cancer), 124 studies were using histological images and 28 were related to omics diagnoses. No study was published to generate a virtual biopsy assessing the presence and severity of organ lesions using non-invasive parameters.

This assertion is confirmed by the results of the European Search carried out by the European Patent Office (EPO) for the application from which the present patent application claims the priority, which are:
an article by Kyung Don YOO et al. whose title is “A Machine Learning Approach Using Survival Statistics to Predict Graft Survival in Kidney Transplant Recipients: A Multicenter Cohort Study”, Scientific Reports, volume 7, number 1;
an article by Derek A DUBAY et al. whose title is “Development and future deployment of a 5 years allograft survival model for kidney transplantation”, Nephrology, volume 24, number 8;
an article by Irina SCHEFFNER et al. whose title is “Patient Survival After Kidney Transplantation: Important Role of Graft-sustaining Factors as Determined by Predictive Modeling Using Random Survival Forest Analysis”, Transplantation, volume 104, number 5;
an article by Vijaya B. KOLACHALAMA et al. whose title is “Association of Pathological Fibrosis With Renal Survival Using Deep Neural Networks”, Kidney International Reports, volume 3, number 2, and
an article by Qiongjing YUAN et al. whose title is “Role of Artificial Intelligence in Kidney Disease”, International Journal of Medical Science, volume 7, number 7.

Thereagain, none of these documents suggests to generate a virtual biopsy assessing the presence and severity of organ lesions using non-invasive parameters.

Added Value of this Study

This study develops and validates the first virtual biopsy system in medicine by using qualified datasets from 17 centers worldwide. The Applicant used commonly assessed clinical and biological parameters to predict and grade specific histological lesions related to tissue fibrosis, arteriosclerosis, arterial hyalinosis and glomerulosclerosis. The Applicant used multiple machine learning algorithms to achieve robust and accurate discrimination and calibration of the derived virtual biopsy system and showed generalizability of the Applicant's results in multiple clinical scenarios.

Implications of all the Available Evidence

This study demonstrates the performance of a non-invasive virtual biopsy system, which is feasible and easily implementable in daily transplant practice and open new avenues for enhanced decision-making process for transplant patient management. To implement this system in routine clinical practice. The Applicant has built an online ready-to-use application that enables clinicians to visualize the virtual biopsy results.

INTRODUCTION

In medicine, biopsy has become a standard test for establishing a diagnosis for both malignant, benign tumors as well as characterizing inflammatory diseases and other pathologic processes, thereby guiding therapeutic management.

In transplant medicine, the biopsy of the organ has been performed since the first pioneering work of Barry et al as well as Hamburger in Paris, becoming the gold standard for diagnosing allograft rejection and other various pathological process that harms the allograft. The histological evaluation of donors also called “day-zero biopsies”, have been also implemented in several transplant programs, to judge the quality of a donor organ and, on occasion, to rule out the possibility of underlying diseases in donors. Besides, day-zero biopsies provide a valuable baseline from which the results of subsequent biopsies of the kidney allograft can be compared to and may also advocate therapeutic strategies.

Despite their potential usefulness, day-zero biopsies are still not performed at many transplant centers and happen only in specific situations, since it remains an invasive, time-consuming and costly procedure that requires organization of surgical, medical, pathological and technical resources. In addition, assessing organ quality has become even more challenging in the current worldwide increase of transplantation from older donors, donation after circulatory death, and donors with significant clinical risk factors. These vulnerable organs may carry, at the time of transplantation arteriosclerosis, fibrosis, hyalinosis and glomerulosclerosis lesions. If revealed in a biopsy of post-transplantation that is not put into perspective with a day-zero biopsy, these histological lesions might be wrongly attributed to calcineurin inhibitor toxicity, infectious diseases or allo-immune response, because of their non-specificity, with significant impact for decision making and patient management.

To circumvent these limitations, the Applicant designed a study to develop and validate a virtual biopsy system that uses routinely collected donor parameters to predict the kidney day-zero biopsy results. Since machine learning has demonstrated its clinical relevance in many medical specialties and superior performance to logistic regression, the Applicant based his analyses on machine learning methods as well as traditional statistical approaches using a large and qualified international cohort of donors who underwent routine and protocolized collection of donor parameters together with day-zero biopsy assessment using the standards of the international Banff allograft histopathology classification.

The final goal of the Applicant was to provide clinicians with a virtual biopsy system to guide diagnostics, therapeutics and immediate patient management post-transplant and to minimize additional risks and costs to perform day-zero biopsies only using standard donor parameters.

METHODS

Study Design and Population

The population consisted in living or deceased donors for kidney transplantation enrolled from Jan. 1, 2000 to Dec. 31, 2019 who underwent kidney biopsies performed prior to kidney transplantation. The study involved 17 institutions from 7 countries in Europe, North America, and Australia. A total of 14,080 kidney biopsies were assessed overall. Exclusion criteria were inadequate biopsies according to Banff international classification requirements (n=1,088, 7.7%). A total of 12,992 kidney allograft biopsies were included for the final analyses.

All data were anonymized, the clinical data collected from each center and entered into the Paris Transplant Group database (French data protection authority (CNIL) registration number: 363505). On Jan. 1, 2020, the data were accessed from the database. The study protocol was approved by the Paris Transplant Group's institutional review board (https://www.paristransplantgroup.org). Written informed consent was given by all living donors at the time of transplantation. At the time of transplantation, all data from the Paris Transplant Group centers (Necker hospital, Saint Louis hospital, Toulouse hospital) were entered prospectively; a structured protocol was used to ensure harmonization across study centers. To ensure data accuracy, data have been sent for an annual audit. As part of standard clinical procedures, other datasets from the European, North American, and Australian centers were compiled, entered in the databases of the centers in accordance with local and national regulatory standards and submitted to the Paris Transplant Group anonymously.

Kidney Biopsy Histological Assessment and Protocols

Day-zero biopsies were performed after the organ was removed from the donor in accordance with the standard practices by a surgeon using a 16-gauge needle device or a straight blade. The tissue was immediately fixed in an alcohol—formalin—acetic acid solution then subsequently embedded in paraffin or immediately frozen. The biopsy sections (4 μm) were stained with periodic acid-Schiff, Masson's trichrome, haematoxylin and eosin. Using the international Banff scores, 19 general pathologists or trained kidney pathologists graded the graft biopsies lesions using the following criteria: glomeruli number, arteriosclerosis, arterial hyalinosis, interstitial fibrosis and tubular atrophy, and the percentage of sclerotic glomeruli. The Banff grading scheme for these lesions are described in detail in Table 6.

A detailed table summarizing other participating centers' biopsy practices and procedures is presented in Table 7.

Outcome of Interest

The outcome of interest was the biopsy result according to the international Banff classification of allograft pathology that uses a validated semi-quantitative ordinal grading scheme for all kidney compartments including: i) arteriosclerosis defined by arterial intimal thickening in the most severely affected artery (Banff “cv” score), ii) arteriolar hyalinosis defined by periodic acid-Schiff (PAS)-positive arteriolar hyaline thickening (Banff “ah” score), and iii) interstitial fibrosis and tubular atrophy (Banff “IFTA” score) computed with the extent of cortical fibrosis (Banff “ci” score) and cortical tubular atrophy (Banff “ct” score). Lastly, the continuous percentage of sclerotic glomeruli defined by the percentage of the total number of glomeruli affected by global sclerosis (Banff “glomerulosclerosis” score). The Banff grading scheme in detail is available in Table 6.

Candidate Predictors of Kidney Biopsy

A total of 11 candidate predictors of kidney day-zero histological lesions was examined, comprising donor's age, sex, type (living or deceased donor), donor's cerebrovascular cause of death, donor after circulatory death (DCD), donor's history of hypertension, diabetes, hepatitis C virus (HCV) status, body mass index (BMI), serum creatinine at donation, and donor proteinuria status.

Statistical Analyses

Descriptive Analyses of Baseline Characteristics

For continuous variables, means and standard deviations (SDs) were used. We compared means and proportions between groups using Student's t-test, analysis of variance (ANOVA) (or Mann-Whitney test and Kruskal-Wallis if appropriate) or the chi-squared test (or Fisher's exact test if appropriate).

Datasets Splitting

The dataset was randomly divided into train (75%) and test (25%) sets for the prediction of the four day-zero histological lesion scores (cv, ah, IFTA, being ordinal variables and glomerulosclerosis, being continuous). The random divisions were stratified by each histological lesion score so that the training and test sets both can share the nearly equally balanced information from them. To minimize the data imbalance in the lesion scores, which had more mild/lower grades than severe/higher grades, we applied an up-sampling method by resampling random values from the mild/lower grade. These datasets preparation and pre-process steps were done with caret R Package. The baseline characteristics of the training and test sets are summarized in Table 8.

Development of the Machine Learning Based Virtual Biopsy System

To develop the virtual biopsy system, probabilities were computed for each day-zero histological lesion score from six machine learning models: random forests (RF), model averaged neural networks (avNNet), gradient boosting machine (GBM), extreme gradient boosting tree (XGBoost), linear discriminant analysis (LDA), and naive Bayes (NB). To compare with machine learning models, traditional multinomial logistic regression (MNOM) was also performed. To avoid overfitting, combinations of hyperparameters were optimized by robust 10-fold cross-validation when tuning the models. In addition, the cross-validation process was repeated three times to minimize sampling bias. Then, the machine learning classification models (base models) were aggregated by averaging probabilities generated by each model to decrease bias and improve predictive performance (ensemble model). For continuous day-zero histological lesion, the percentage of glomerulosclerosis, an ensemble model was created using a linear regression of regression models (base models) to enhance performance, with 3-times repeated 10-fold cross-validation. The Applicant refrained from performing MNOM, LDA, and NB to predict the glomerulosclerosis lesion because they are exclusively designed to predict categorical variables (classification).

Machine Learning Prediction Performances

To assess the discrimination performance of the machine learning models used for glomerulosclerosis, which is continuous, we used the mean absolute error (MAE). For ordinal day-zero histological lesion scores, arteriosclerosis (cv), arteriolar hyalinosis (ah), and interstitial fibrosis and tubular atrophy (IFTA), we used multi-area under curve (multi-AUC) of unweighted pairwise discriminability of histological lesion scores by Hand and Till's formula. Model calibration was examined with confusion matrices. Furthermore, to test the robustness of the prediction performances, we introduced artificial random errors on our test sets then gradually increased the number of errors and examined how the prediction performances of our ensemble models were affected.

Multiple Imputation of Missing Data

For biopsies with at least one missing data element for predictors of interest, random forest imputation algorithm was performed using the missForest R package. The maximum number of iterations was set to 10 times for multiple imputation.

The missing values were imputed with random forest algorithm, which was implemented in missForest R package. The donor parameters and biopsy findings used in the imputation algorithm were i) age, ii) sex, iii) donor type (living or deceased donor), iv) cerebrovascular cause of death, v) donor after circulatory death (DCD), vi) history of hypertension, vii) diabetes, viii) hepatitis C virus (HCV) status, ix) body mass index (BMI), x), kidney function defined by serum creatinine, xi) proteinuria status, xii) arteriosclerosis (Banff cv score), xiii) arteriolar hyalinosis (Banff ah score), xiv) interstitial fibrosis and tubular atrophy (Banff IFTA score), xv) percentage of sclerotic glomeruli (Banff glomerulosclerosis score). The maximum number of iterations was set to 10 times.

The detail of the imputation process and results are presented in Table 9.

Software

Descriptive analyses and machine learning analyses were conducted using STATA (version 15, Data Analysis and Statistical Software) and R (version 3.5.1, R Foundation for Statistical Computing).

### RESULTS

Characteristics of the Cohort

A total of 12,992 day-zero biopsies were included from the 17 participating transplant centers from Jan. 1, 2000 to Dec. 31, 2019, of whom 5,905 biopsies (45.45%) were from the 10 European centers, 6,663 (51.29%) from the six North American centers, and 424 (3.26%) from the Australian center. The mean donor age was 49.75±15.13 years. 6,082 (46.85%) were female, and 9,449 (72.73%) were deceased donors. The mean serum creatinine was 1.07±0.74 mg/dl. Baseline donor characteristics and comparison among European, North American and Australian cohorts are presented in detail in Table 1.

In more details for the population, the European centers included Necker Hospital, Paris, France (n=1218), Saint-Louis Hospital, Paris, France (n=856), Toulouse Hospital, Toulouse, France (n=522), Bicêtre Hospital, Kremlin Bicêtre, France (n=575), University Hospital, Leuven, Belgium (n=915), University Hospital, Liege, Belgium (n=130), University Hospital Centre Zagreb, Zagreb, Croatia (n=566), Hospital Clinic i Provincial de Barcelona, Barcelona, Spain (n=486), Hospital Vall d'Hebrón, Barcelona, Spain (n=454), and Bellvitge University Hospital, Barcelona, Spain (n=183). The North American centers included the Mayo Clinic, Rochester, Minn. (n=2922), the Mayo Clinic, Phoenix, Ariz. (n=92), Columbia University Medical Center, New York, N.Y., USA (n=871), University of British Columbia, Vancouver, Canada (n=465), University of Alberta, Edmonton, Canada (n=1226), and United Network Organ for Organ Sharing (UNOS, n=1087). The Australian center included the Royal Adelaide Hospital, Adelaide, Australia (n=424).

Baseline characteristics of the donors stratified by country and center are also presented in the Tables 3, 4.1 and 4.2.

Kidney Biopsy Results

Table 1 depicts the day-zero kidney biopsy results stratified by European, North American and Australian cohorts. The mean percentage of glomerulosclerosis was of 7.67%±10.87 (8.39%±11.28 among deceased donors). The arteriosclerosis (cv) lesion score's distribution was 52.32%, 29.76%, 16.05%, and 1.87% for Banff scores 0, 1, 2, and 3, respectively. The arteriolar hyalinosis (ah) lesion score's distribution was 61.57%, 26.93%, 9.58% and 1.91% for scores 0, 1, 2, 3, respectively. Finally, the interstitial fibrosis and tubular atrophy (IFTA) lesion score's distribution was 60.19%, 31.29%, 8.00% and 0.52% for scores 0, 1, 2, and 3, respectively. Most moderate or severe (score 2 or 3) lesions were from deceased donors (see Table 5).

Virtual Biopsy System Development Using Machine Learning

The dataset was randomly divided into train (75%) and test (25%) sets for the prediction of the four day-zero histological lesion scores. The comparison between the training and test sets are shown in the Tables 8, 8.1, 8.2, 8.3 and 8.4.

Multiple machine learning models were generated for the biopsy lesion scores including arteriosclerosis (cv), arteriolar hyalinosis (ah), interstitial fibrosis and tubular atrophy (IFTA), and glomerulosclerosis with the assessment of the donor's characteristics in the training set using the following 11 predictors: age, sex, donor type (living or deceased donor), donor after cerebrovascular death, donor after circulatory death, history of hypertension, diabetes, HCV status, BMI, serum creatinine, and proteinuria status.

Then an ensemble model that groups several machine learning models together to improve performance was generated. We selected the ensemble models with averaging the score probabilities method for ordinal arteriosclerosis (cv), arteriolar hyalinosis (ah), and interstitial fibrosis and tubular atrophy (IFTA) day-zero lesion scores and the best performing model during the cross-validation for the percentage of glomerulosclerosis as virtual biopsy system.

Prediction Model Performance

The ensemble models attained multi-AUCs in the test sets of: 0.738, 0.817, 0.788 for arteriosclerosis (cv), arteriolar hyalinosis (ah), and interstitial fibrosis and tubular atrophy (IFTA), respectively (Table 2). Random forest model performed the best during the cross-validation for the glomerulosclerosis lesion, with a mean absolute error (MAE) of 4.766 in the test set. Table 2 summarizes the performances of all generated models. For all ordinal lesion scores, ensemble models were the best performing models. For glomerulosclerosis lesion, XGBoost model achieved the best discrimination with a MAE of 4.703 in the test set. Calibration is shown as confusion matrices in Tables 10, 10.1 and 10.2.

Donor Parameters Relative Importance on Lesion Scores Prediction

The importance of the 11 donor parameters S used in the ensemble models were examined on each training set. The three most important parameters predictive of the biopsy lesions were: age, serum creatinine, and body mass index (BMI) for arteriosclerosis (cv) and arteriolar hyalinosis (ah), and were age, creatinine, and the history of hypertension for interstitial fibrosis and tubular atrophy (IFTA) and glomerulosclerosis.

Building the Virtual Biopsy Online Application for the Clinicians

The Applicant constructed a ready-to-use online application to offer clinicians an open access to our virtual day-zero biopsy system. The application allows clinicians to enter a single patient's data such as the basic demographics, past medical history, comorbidities, clinical parameters, biological parameters including kidney function, and proteinuria level of a given donor to get i) the corresponding probabilities of belonging to each day-zero histological lesion scores, ii) the corresponding visualization with radar chart.

### SENSITIVITY ANALYSES

Various sensitivity analyses were performed to further increase the robustness of our results and the generalizability of the models.

Validation of the Virtual Biopsy System in Different Subpopulations and Clinical Scenarios

The robustness of the virtual biopsy system was confirmed in different subpopulations and clinical scenarios in test sets, including: i) continent, ii) donor type (living or deceased donor), iii) ethnicity (African American or non-African American donor), and iv) biopsy type (preimplantation or postreperfusion day-zero biopsy) (see Table 11).

Performances of Machine Learning Models Compared with Traditional Multinomial Logistic Regression

The performances of machine learning models were compared with multinomial logistic regression and confirmed the machine learning models, especially tree-based models (e.g. random forest), outperformed classical multinomial logistic regression models (Table 2).

Additional Analyzes to Confirm the Robustness of the Results

The robustness of the virtual biopsy system was assessed on the test sets by generating artificial errors on histological lesion scores to observe any drastic changes of our ensemble models. The Applicant confirmed that the incremental generated errors on the outcome measure (7.64%, 6.87%, 7.22%, respectively, on average per step) was accompanied by a sharp and constant decrease in the classifier's performance.

### DISCUSSION

In this large international cohort study of kidney transplant biopsies from 17 worldwide centers, we derived and validated a novel virtual biopsy system that uses non-invasive and routinely collected donor parameters to predict histological lesions. The virtual biopsy system was developed with an ensemble of machine learning methods to maximize the prediction performances. Overall, it showed good discrimination, calibration, robustness and generalizability in various regions and clinical scenarios. In particular, the virtual biopsy system not only predicts the presence of lesions (binary classification) but also predicts the spectrum of the kidney allograft (multiclass classification), which fosters a more complete clinical interpretation.

Over the past decade, the use of kidneys from older donors with comorbidities expanded the pool of kidneys, raising the question whether pathological examination of donated kidneys could help better characterize organ quality or drive inefficiencies in organ allocation. In addition, many centers are discouraged to perform day-zero biopsy because it remains an invasive and time-consuming procedure that could increase cold ischemia time.

The Applicant believes that the virtual biopsy system has many potential implications:

First, this virtual biopsy system can assist a physician to evaluate and contextualize post-transplant lesions, which might be inherited from the donor; this could reinforce precision medicine and patient monitoring by evaluating whether the chronic lesions are created from immunosuppressive toxicity or donors.

Second, it may be attractive for clinical trials, by helping to improve the randomization of the patients at the time of transplantation using not only the baseline characteristics but also the chronic lesions of kidney donors to avoid selection bias. Moreover, the efficacy of a new treatment is very often based on protocol biopsies where chronic lesions such as fibrosis and arteriosclerosis can be found. Because antibody-mediated rejection or immunosuppressive toxicity can induce those lesion, knowing their origin—whether they were inherited from the donor or from the consequence of the treatment inefficacy—is crucial to avoid misinterpretation of the results and loss of potential useful treatments.

Third, although the rapid improvements in computing power and huge digitized medical history records have led many researchers to attempt integrative approaches to scrutinize unknown fields of medicine using machine learning, it is still difficult to approach these tools in real life for health professionals. Therefore, the easy-to-use online application we generated to support the clinicians to reinforce applicability is an essential aspect of this interdisciplinary study.

Lastly, the idea of virtual biopsy system, using routinely accessible donor parameters to predict biopsy results with the power of machine learning algorithms, can be easily cross-fertilized in other fields of medicine, with a comparable need to predict specific lesions for an enhanced interpretation of the patient prognosis.

### LIMITATIONS

The present study has several limitations. First, ordinal day-zero histological lesions, arteriosclerosis (cv), arteriolar hyalinosis (ah), and interstitial fibrosis and tubular atrophy (IFTA) are imbalanced in our dataset. Although this is common in real life, the consequence is that the models have stronger power to correctly predict the most represented classes (lower histological lesion scores) than the least represented ones (higher histological lesion scores). To circumvent this as best as possible, the Applicant used an up-sampling method, which randomly resamples minor classes, to amplify the power of predicting minor classes to even out the balance among major and minor lesion classes. Additionally, up-sampling method makes the cross-validation overfitting although this only negligibly affects the final discrimination on test sets. Lastly, the Applicant's ensemble models are complex and may require dozens of hours to reproduce. However, the online application offers a real-time assessment of the virtual biopsy.

### CONCLUSION

In summary, the Applicant has derived for the first time a machine learning-based virtual kidney allograft biopsy system that uses easily accessible donor parameters at the time of transplantation. The virtual biopsy system demonstrates accurate performances and robustness across 17 geographically distinct centers and in many clinical scenarios. This system can provide clinicians with a reliable estimation of the day-zero biopsy results, which may reduce cost on invasive and time-consuming procedures, and help guide further biopsies interpretations and patient management.

### APPENDIX

In the appendix, the different tables, which were cited before, are detailed.

TABLE 1
Baseline donor characteristics of population cohort by continent

| | | Overall | | Europe | | North America | | Australia | |
|---|---|---|---|---|---|---|---|---|---|
| | N | (n = 12,992) | N | (n = 5,905) | N | (n = 6,663) | N | (n = 424) | p-value |
| Age (years), mean (SD) | 12992 | 49.8 (15.1) | 5905 | 54.8 (15.4) | 666 | 45.3 (13.4) | 424 | 48.7 (14.6) | <0.001 |
| Sex female, No. (%) | 12981 | 6082 (46.9%) | 5904 | 2489 (42.2%) | 666 | 3392 (50.9%) | 414 | 201 (48.6%) | <0.001 |
| Donor type | | | | | | | | | |
| Deceased donor, No. (%) | 12992 | 9449 (72.7%) | 5905 | 5838 (98.9%) | 666 | 3274 (49.1%) | 424 | 337 (79.5%) | <0.001 |
| Death from circulatory disease, | 12947 | 1144 (12.1%) | 5905 | 772 (13.2%) | 6619 | 305 (9.3%) | 423 | 67 (19.9%) | <0.001 |
| No. (%*) | | | | | | | | | |
| Death from cerebrovascular | 12441 | 4936 (52.2%) | 5905 | 3608 (61.8%) | 6204 | 1174 (35.9%) | 332 | 154 (45.7%) | <0.001 |
| disease, No. (%*) | | | | | | | | | |
| Diabetes mellitus, No. (%) | 11003 | 894 (8.1%) | 4847 | 453 (9.3%) | 573 | 425 (7.4%) | 420 | 16 (3.8%) | <0.001 |
| Hypertension, No. (%) | 11850 | 3275 (27.6%) | 5702 | 2047 (35.9%) | 572 | 1156 (20.2%) | 420 | 72 (17.1%) | <0.001 |
| BMI (kg/m²), mean (SD) | 11920 | 26.8 (5.3) | 5684 | 25.9 (4.6) | 593 | 27.7 (5.8) | 303 | 26.9 (5.6) | <0.001 |
| HCV status, No. (%) | 12540 | 114 (0.9%) | 5719 | 42 (0.7%) | 639 | 72 (1.1%) | 422 | 0 (0.0%) | 0.007 |
| Creatinine (mg/dL), mean (SD) | 10912 | 1.1 (0.7) | 5570 | 1.0 (0.5) | 493 | 1.2 (0.9) | 409 | 0.8 (0.3) | <0.001 |
| Proteinuria, No. (%) | 9288 | 2798 (30.1%) | 4070 | 1705 (41.9%) | 513 | 1090 (21.2%) | 83 | 3 (3.6%) | <0.001 |
| Number of Glomeruli, mean (SD) | 7610 | 34.1 (29.8) | 3840 | 26.9 (22.7) | 334 | 41.8 (35.5) | 421 | 38.5 (18.3) | <0.001 |
| Arteriosclerosis (cv) Banff score, No. (%) | 12381 | | 5793 | | 6405 | | 183 | | <0.001 |
| 0 | | 6478 (52.3%) | | 3028 (52.3%) | | 3361 (52.5%) | | 89 (48.6%) | |
| 1 | | 3684 (29.8%) | | 1849 (31.9%) | | 1772 (27.7%) | | 63 (34.4%) | |
| 2 | | 1987 (16.1%) | | 786 (13.6%) | | 1181 (18.4%) | | 20 (10.9%) | |
| 3 | | 232 (1.9%) | | 130 (223%) | | 91 (1.4%) | | 11 (6.0%) | |
| Arteriolar hyalinosis (ah) Banff score, | 11487 | 5641 | | 5439 | | 407 | | | |
| No. (%) | | | | | | | | | |
| 0 | 7073 (61.6%) | 3051 (54.2%) | | 3701 (68.0%) | | 321 (78.9%) | | <0.001 | |
| 1 | 3094 (26.9%) | 11815 (32.1%) | | 1208 (22.2%) | | 71 (17.4%) | | | |
| 2 | 1101 (9.6%) | 626 (11.1%) | | 463 (8.5%) | | 12 (2.9%) | | | |
| 3 | 219 (1.9%) | 149 (2.7%) | | 67 (1.2%) | | 3 (0.7%) | | | |
| Interstitial fibrosis and tubular atrophy | 12795 | 5876 | | 6495 | | 424 | | <0.001 | |
| (IFTA) Banff score, No. (%) | | | | | | | | | |
| 0 | 07701 (60.2%) | 3276 (55.8%) | | 4049 (62.3%) | | 376 (88.7%) | | | |
| 1 | 4004 (31.3%) | 2269 (38.6%) | | 1693 (26.1%) | | 42 (9.9%) | | | |
| 2 | 1023 (8.0%) | 278 (4.7%) | | 740 (11.4%) | | 5 (1.2%) | | | |
| 3 | 67 (0.5%) | 53 (0.9%) | | 13 (0.2%) | | 1 (0.2%) | | | |
| Glomerulosclerosis, mean (SD) | 7517 | 7.7 (10.9) | 5197 | 8.6 (11.4) | 189 | 5.0 (8.7) | 423 | 7.7 (10.5) | <0.001 |

Abbreviations: BMI: body mass index. HCV: hepatitis C virus.
*% was calculated among deceased donors.
Proteinuria values were positive when dipstick greater than or equal to 1 or urine protein to creatinine ratio (UPCR, g/g) greater than or equal to 0.5 g/g.

TABLE 2
Base machine learning classifiers and ensemble models’ performances in test sets
The models used for ordinal scores (multiclass classification) are as follows: random forest,
gradient boosting machine, extreme gradient boosting tree, naive bayes, linear discriminant
analysis, multinomial logistic regression, and model averaged neural network. The models
used for the percentage of glomerulosclerosis (regression) are as follows: random forest,
gradient boosting machine, extreme gradient boosting tree, and model averaged neural network.
We also created ensemble models (aggregated 6 machine learning models except for the
multinomial logistic regression); for the ordinal day-zero lesion scores, we averaged the
probabilities; for the percentage of glomerulosclerosis, we used linear regression of the
models we created. For the ordinal day-zero lesion scores, model performances
were assessed by Hand and Till’s area under the curve (multi-AUC). For the percentage
of glomerulosclerosis, model performances were assessed by mean absolute error (MAE).

| | | | | Mean Absolute |
|---|---|---|---|---|
| | Hand and Till’s Multi-AUC | | | Error |
| | | Arteriolar | Interstitial fibrosis | (MAE) |
| | Arteriosclerosis | hyalinosis | tubular atrophy | Glomerulosclerosis |
| Machine learning Model | (cv Banff score) | (ah Banff score) | (IFTA Banff score) | in percentage |
| Machine Learning Models | | | | |
| Random Forest | 0.708 | 0.806 | 0.754 | 4.766 |
| Gradient Boosting Machine | 0.709 | 0.790 | 0.719 | 4.923 |
| Extreme Gradient Boosting Tree | 0.719 | 0.805 | 0.778 | 4.703 |
| Naive Bayes* | 0.691 | 0.746 | 0.751 | —* |
| Linear Discriminant Analysis* | 0.691 | 0.746 | 0.732 | —* |
| Model Averaged Neural Network | 0.679 | 0.740 | 0.713 | 7.37 |
| Ensemble Model | 0.738 | 0.817 | 0.788 | 4.748 |
| Traditional Statistical Model | | | | |
| Multinomial Logistic Regression* | 0.694 | 0.743 | 0.731 | —* |

Abbreviation: area under the curve (AUC, higher the better), mean absolute error (MAE, lower the better).
Ensemble models for cv, ah, IFTA and random forest that showed the best performance in cross-validation for the percentage of glomerulosclerosis were selected as virtual biopsy system.
*Naive bayes, linear discriminant analysis, and multinomial logistic regression are not developed for regression problem but for classification

TABLE 3
Baseline donor characteristics of the population cohort by country

| | Overall | Australia | Belgium | Canada | Croatia | Spain | France | USA |
|---|---|---|---|---|---|---|---|---|
| | (n = 12,992) | (n = 424) | (n = 1,045) | (n = 1,691) | (n = 566) | (n = 1,123) | (n = 3,171) | (n = 4,972) |
| Age (years), | 49.751 | 48.703 | 48.879 | 42.883 (13.750) | 49.657 (11.972) | 63.340 (11.173) | 54.642 (16.481) | 46.181 (13.203) |
| mean (SD) | (15.134) | (14.622) | (13.462) | | | | | |
| Sex female, | 6082 | 201 | 473 | 808 (47.782%) | 237 (41.873%) | 422 (37.578%) | 1357 (42.808%) | 2584 (51.971%) |
| No. (%) | (46.853%) | (48.551%) | (45.263%) | | | | | |
| Donor type | | | | | | | | |
| Deceased donor, | 9449 | 337 | 1045 | 1156 (68.362%) | 566 (100.000%) | 1123 (100.000%) | 3104 (97.887%) | 2118 (42.599%) |
| No. (%) | (72.729%) | (79.481%) | (100.000%) | | | | | |
| Death from | 1144 | 67 | 226 | 130 (11.246%) | 0 (0.000%) | 338 (30.098%) | 208 (6.701%) | 175 (8.263%) |
| circulatory | (12.107%) | (19.881%) | (21.627%) | | | | | |
| disease, No. | | | | | | | | |
| (%*) | | | | | | | | |
| Death from | 4936 | 154 | 590 | 391 (33.824%) | 384 (67.845%) | 789 (70.258%) | 1845 (59.439%) | 783 (36.969%) |
| cerebrovascular | (52.238%) | (45.697%) | (56.459%) | | | | | |
| disease, No. | | | | | | | | |
| (%*) | | | | | | | | |
| Diabetes | 894 | 16 | 4 (3.810%) | 60 (4.231%) | 16 (2.827%) | 175 (15.909%) | 258 (8.388%) | 365 (8.453%) |
| mellitus, | (8.125%) | (3.810%) | | | | | | |
| No. (%) | | | | | | | | |
| Hypertension, | 3275 | 72 | 223 | 196 (13.940%) | 212 (37.456%) | 661 (60.091%) | 951 (31.584%) | 960 (22.212%) |
| No. (%) | (27.637%) | (17.143%) | (21.756%) | | | | | |
| BMI (kg/m²), | 26.818 | 26.945 | 25.511 | 26.491 (5.383) | 26.367 (3.787) | 27.520 (4.817) | 25.422 (4.696) | 28.162 (5.830) |
| mean (SD) | (5.312) | (5.635) | (4.151) | | | | | |
| HCV status, | 114 | 0 (0.000%) | 0 (0.000%) | 16 (1.043%) | 0 (0.000%) | 4 (0.361%) | 38 (1.266%) | 56 (1.151%) |
| No. (%) | (0.909%) | | | | | | | |
| Creatinine | 1.071 | 0.786 | 0.787 (0.450) | 0.958 (0.642) | 0.936 (0.400) | 1.007 (0.481) | 1.003 (0.575) | 1.343 (0.995) |
| (mg/dL), | (0.737) | (0.303) | | | | | | |
| mean (SD) | | | | | | | | |
| Proteinuria, | 2798 | 3 (3.614%) | 52 (44.828%) | 299 (36.823%) | 123 (21.731%) | 120 (19.108%) | 1410 (51.087%) | 791 (18.297%) |
| No. (%) | (30.125%) | | | | | | | |
| Number of | 34.120 | 38.463 | 27.731 | 32.590 (24.706) | 54.272 (32.991) | N/A | 21.955 (16.264) | 51.034 (41.665) |
| Glomeruli, | (29.780) | (18.285) | (16.962) | | | | | |
| mean (SD) | | | | | | | | |
| Arteriosclerosis | | | | | | | | |
| (cv) Banff score, | | | | | | | | |
| No. (%) | | | | | | | | |
| 0 | 6478 | 89 | 924 | 1069 (72.672%) | 410 (72.438%) | 508 (45.317%) | 1186 (38.746%) | 2292 (46.453%) |
| | (52.322%) | (48.634%) | (88.421%) | | | | | |
| 1 | 3684 | 63 | 96 (9.187%) | 286 (19.443%) | 145 (25.618%) | 546 (48.707%) | 1062 (34.695%) | 1486 (30.118%) |
| | (29.755%) | (34.426%) | | | | | | |
| 2 | 1987 | 20 | 24 (2.297%) | 107 (7.274%) | 7 (1.237%) | 63 (5.620%) | 692 (22.607%) | 1074 (21.767%) |
| | (16.049%) | (10.929%) | | | | | | |
| 3 | 232 | 11 | 1 (0.096%) | 9 (0.612%) | 4 (0.707%) | 4 (0.357%) | 121 (3.953%) | 82 (1.662%) |
| | (1.874%) | (6.011%) | | | | | | |
| Arteriolar | | | | | | | | |
| hyalinosis (ah) | | | | | | | | |
| Banff score, | | | | | | | | |
| No. (%) | | | | | | | | |
| 0 | 7073 | 321 | 774 | 872 (53.009%) | 349 (61.661%) | 591 (63.961%) | 1337 (43.004%) | 2829 (74.565%) |
| | (61.574%) | (78.870%) | (74.280%) | | | | | |
| 1 | 3094 | 71 | 211 | 438 (26.626%) | 190 (33.569%) | 274 (29.654%) | 1140 (36.668%) | 770 (20.295%) |
| | (26.935%) | (17.445%) | (20.250%) | | | | | |
| 2 | 1101 | 12 | 53 (5.086%) | 291 (17.690%) | 24 (4.240%) | 57 (6.169%) | 492 (15.825%) | 172 (4.533%) |
| | (9.585%) | (2.948%) | | | | | | |
| 3 | 219 | 3 | 4 (0.384%) | 44 (2.675%) | 3 (0.530%) | 2 (0.216%) | 140 (4.503%) | 23 (0.606%) |
| | (1.907%) | (0.737%) | | | | | | |
| Interstitial | | | | | | | | |
| fibrosis | | | | | | | | |
| and tubular | | | | | | | | |
| atrophy (IFTA) | | | | | | | | |
| Banff score, | | | | | | | | |
| No. (%) | | | | | | | | |
| 0 | 7701 | 376 | 763 | 894 (58.165%) | 228 (40.283%) | 316 (28.164%) | 1969 (62.647%) | 3155 (63.635%) |
| | (60.188%) | (88.679%) | (73.014%) | | | | | |
| 1 | 4004 | 42 | 257 | 589 (38.321%) | 304 (53.710%) | 755 (67.291%) | 953 (30.321%) | 1104 (22.267%) |
| | (31.293%) | (9.906%) | (24.593%) | | | | | |
| 2 | 1023 | 5 | 21 (2.010%) | 52 (3.383%) | 33 (5.830%) | 50 (4.456%) | 174 (5.536%) | 688 (13.877%) |
| | (7.995%) | (1.179%) | | | | | | |
| 3 | 67 | 1 | 4 (0.383%) | 2 (0.130%) | 1 (0.177%) | 1 (0.089%) | 47 (1.495%) | 11 (0.222%) |
| | (0.524%) | (0.236%) | | | | | | |
| Glomerulosclerosis, | 7.666 | 7.732 | 6.606 | 7.029 (8.641) | 6.415 (8.988) | 7.643 (7.763) | 9.871 (12.126) | 4.305 (8.633) |
| mean (SD) | (10.874) | (10.520) | (11.320) | | | | | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.
*% was calculated among deceased donors.

TABLE 4.1
Baseline donor characteristics of the population cohort by center

| | | | | | | | Columbia | | |
|---|---|---|---|---|---|---|---|---|---|
| | | Mayo | University | | | | University | | |
| | | Clinic | of | Necker | | KU | Medical | Saint Louis | Bicêtre |
| | Overall | Rochester | Alberta | hospital | UNOS | LEUVEN | Center | hospital | hospital |
| | (n = 12,992) | (n = 2922) | (n = 1226) | (n = 1218) | (n = 1087) | (n = 915) | (n = 871) | (n = 856) | (n = 575) |
| Age (years), | 49.751 | 43.543 | 41.219 | 55.273 | 55.494 | 49.192 | 43.614 | 50.814 (15.599) | 61.319 (16.113) |
| mean (SD) | (15.134) | (12.490) | (13.345) | (16.865) | (10.937) | (13.610) | (12.760) | | |
| Sex female, No. | 6082 | 1611 | 598 | 526 | 517 | 415 | 417 | 335 (39.136%) | 235 (40.870%) |
| (%) | (46.853%) | (55.133%) | (48.777%) | (43.221%) | (47.562%) | (45.355%) | (47.876%) | | |
| Donor type | | | | | | | | | |
| Deceased donor, | 9449 | 552 | 810 | 1218 | 1087 | 915 | 399 | 856 (100.000%) | 575 (100.000%) |
| No. (%) | (72.729%) | (18.891%) | (66.069%) | (100.000%) | (100.000%) | (100.000%) | (45.809%) | | |
| Death from | 1144 | 0 (0.000%) | 26 | 27 | 140 | 178 | 35 | 78 (9.112%) | 103 (17.913%) |
| circulatory | (8.836%) | | (2.121%) | (2.217%) | (12.879%) | (19.454%) | (4.018%) | | |
| disease, No. (%*) | | | | | | | | | |
| Death from | 4936 | 0 (0.000%) | 285 | 732 | 605 | 517 | 178 | 517 (60.397%) | 326 (56.696%) |
| cerebrovascular | (39.675%) | | (35.185%) | (60.099%) | (55.658%) | (56.503%) | (20.436%) | | |
| disease, No. (%*) | | | | | | | | | |
| Diabetes mellitus, | 894 | 0 (0.000%) | 36 | 102 | 317 | N/A | 48 | 48 (5.701%) | 80 (14.035%) |
| No. (%) | (8.125%) | | (3.666%) | (8.793%) | (29.599%) | | (5.549%) | | |
| Hypertension, | 3275 | 0 (0.000%) | 130 | 360 | 792 | 190 | 168 | 221 (26.341%) | 254 (44.640%) |
| No. (%) | (27.637%) | | (13.306%) | (30.901%) | (73.950%) | (20.765%) | (19.333%) | | |
| BMI (kg/m²), | 26.818 | 27.672 | 26.156 | 25.215 | 29.872 | 25.436 | 27.349 | 25.169 (4.556) | 26.103 (5.035) |
| mean (SD) | (5.312) | (5.003) | (5.382) | (4.739) | (7.361) | (4.108) | (5.279) | | |
| HCV status, | 114 | 4 (0.138%) | 7 (0.655%) | 27 | 24 | 0 | 27 | 2 (0.234%) | 3 (0.522%) |
| No. (%) | (0.909%) | | | (2.220%) | (2.208%) | (0.000%) | (3.100%) | | |
| Creatinine | 1.071 | 0.940 | 0.923 | 1.042 | 1.892 | 0.792 | 1.630 | 0.993 (0.512) | 0.969 (0.482) |
| (mg/dL), | (0.737) | (0.206) | (0.561) | (0.668) | (1.328) | (0.466) | (1.103) | | |
| mean (SD) | | | | | | | | | |
| Proteinuria, | 2798 | 0 (0.000%) | 134 | 670 | 590 | N/A | 201 | 320 (41.078%) | 281 (56.998%) |
| No. (%) | (30.125%) | | (34.805%) | (57.118%) | (54.731%) | | (23.291%) | | |
| Number of | 34.120 | 20.700 | 35.084 | 18.488 | 67.423 | N/A | N/A | 19.033 (7.298) | 38.975 (29.976) |
| Glomeruli, | (29.780) | (7.935) | (27.738) | (7.786) | (43.188) | | | | |
| mean (SD) | | | | | | | | | |
| Arteriosclerosis | | | | | | | | | |
| (cv) Banff score, | | | | | | | | | |
| No. (%) | | | | | | | | | |
| 0 | 6478 | 1739 | 644 | 369 | 263 | 804 | 235 | 305 (37.105%) | 231 (41.924%) |
| | (52.322%) | (60.194%) | (64.016%) | (30.982%) | (24.195%) | (87.869%) | (27.074%) | | |
| 1 | 3684 | 936 | 261 | 422 | 159 | 90 | 361 | 289 (35.158%) | 209 (37.931%) |
| | (29.755%) | (32.399%) | (25.944%) | (35.432%) | (14.627%) | (9.836%) | (41.590%) | | |
| 2 | 1987 | 207 | 94 | 352 | 639 | 21 | 224 | 197 (23.966%) | 88 (15.971%) |
| | (16.049%) | (7.165%) | (9.344%) | (29.555%) | (58.786%) | (2.295%) | (25.806%) | | |
| 3 | 232 | 7 (0.242%) | 7 (0.696%) | 48 | 26 | 0 | 48 | 31 (3.771%) | 23 (4.174%) |
| | (1.874%) | | | (4.030%) | (2.392%) | (0.000%) | (5.530%) | | |
| Arteriolar | | | | | | | | | |
| hyalinosis (ah) | | | | | | | | | |
| Banff score, | | | | | | | | | |
| No. (%) | | | | | | | | | |
| 0 | 7073 | 2390 | 648 | 416 | N/A | 663 | 363 | 342 (40.235%) | 314 (58.148%) |
| | (61.574%) | (84.363%) | (54.915%) | (34.437%) | | (72.538%) | (41.724%) | | |
| 1 | 3094 | 391 | 349 | 493 | N/A | 202 | 369 | 349 (41.059%) | 138 (25.556%) |
| | (26.935%) | (13.802%) | (29.576%) | (40.811%) | | (22.101%) | (42.414%) | | |
| 2 | 1101 | 50 | 166 | 241 | N/A | 46 | 119 | 120 (14.118%) | 57 (10.556%) |
| | (9.585%) | (1.765%) | (14.068%) | (19.950%) | | (5.033%) | (13.678%) | | |
| 3 | 219 | 2 (0.071%) | 17 | 58 | N/A | 3 | 19 | 39 (4.588%) | 31 (5.741%) |
| | (1.907%) | | (1.441%) | (4.801%) | | (0.328%) | (2.184%) | | |
| Interstitial fibrosis | | | | | | | | | |
| and tubular atrophy | | | | | | | | | |
| (IFTA) Banff | | | | | | | | | |
| score, No. (%) | | | | | | | | | |
| 0 | 7701 | 2302 | 609 | 832 | 170 | 645 | 621 | 555 (64.912%) | 177 (31.107%) |
| | (60.188%) | (79.161%) | (56.810%) | (69.507%) | (15.639%) | (70.492%) | (71.297%) | | |
| 1 | 4004 | 598 | 413 | 297 | 236 | 246 | 242 | 239 (27.953%) | 306 (53.779%) |
| | (31.293%) | (20.564%) | (38.526%) | (24.812%) | (21.711%) | (26.885%) | (27.784%) | | |
| 2 | 1023 | 6 (0.206%) | 48 | 56 | 673 | 20 | 7 | 47 (5.497%) | 67 (11.775%) |
| | (7.995%) | | (4.478%) | (4.678%) | (61.914%) | (2.186%) | (0.804%) | | |
| 3 | 67 | 2 (0.069%) | 2 | 2 | 8 | 4 | 1 | 14 (1.637%) | 19 (3.339%) |
| | (0.524%) | | (0.187%) | (1.003%) | (0.736%) | (0.437%) | (0.115%) | | |
| Glomerulosclerosis, | 7.666 | 5.641 | N/A | 8.986 | N/A | 6.661 | 3.630 | 9.482 (13.168) | 12.266 (12.527) |
| mean (SD) | (10.874) | (8.546) | | (11.193) | | (11.051) | (8.847) | | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.
*% was calculated among deceased donors.

TABLE 4.2
Baseline donor characteristics of the population cohort by center

| | | | | Hospital | | | | | | |
|---|---|---|---|---|---|---|---|---|---|---|
| | | University | Centre | Clinic i | University | Vall | | | Centre | |
| | | Hospital | Hospitalier | Provincial | of | d'Hebron | Royal | Hospital | hospitalier | Mayo |
| | Overall | Centre | Universitaire | de | British | University | Adelaide | Universitari | universitaire | Clinic |
| | (n = | Zagreb | de Toulouse | Barcelona | Columbia | Hospital | Hospital | de Bellvitge | de Liège | Phoenix |
| | 12,992) | (n = 566) | (n = 522) | (n = 486) | (n = 465) | (n = 454) | (n = 424) | (n = 183) | (n = 130) | (n = 92) |
| Age (years), | 49.751 | 49.657 | 52.094 | 62.035 | 47.268 | 63.219 | 48.703 | 67.104 | 46.677 | 44.228 |
| mean (SD) | (15.134) | (11.972) | (14.905) | (10.280) | (13.852) | (11.688) | (14.622) | (11.365) | (12.196) | (14.271) |
| Sex female, | 6082 | 237 | 261 | 154 | 210 | 196 | 201 | 72 | 58 | 39 |
| No. (%) | (46.853%) | (41.873%) | (50.000%) | (31.687%) | (45.161%) | (43.172%) | (48.551%) | (39.344%) | (44.615%) | (42.391%) |
| Donor type | | | | | | | | | | |
| Deceased | 9449 | 566 | 455 | 486 | 346 | 454 | 337 | 183 | 130 | 80 |
| donor, No. (%) | (72.729%) | (100.000%) | (87.165%) | (100.000%) | (74.409%) | (100.000%) | (79.481%) | (100.000%) | (100.000%) | (86.957%) |
| Death from | 1144 | 0 | 0 | 195 | 104 | 104 | 67 | 39 | 48 | 0 |
| circulatory | (8.836%) | (0.000%) | (0.000%) | (40.123%) | (22.366%) | (22.907%) | (15.839%) | (21.311%) | (36.923%) | (0.000%) |
| disease, No. (%) | | | | | | | | | | |
| Death from | 4936 | 384 | 270 | 388 | 106 | 289 | 154 | 112 | 73 | 0 |
| cerebrovascular | (39.675%) | (67.845%) | (51.724%) | (79.835%) | (22.796%) | (63.656%) | (46.386%) | (61.202%) | (56.154%) | (0.000%) |
| disease, No. | | | | | | | | | | |
| (%) | | | | | | | | | | |
| Diabetes | 894 | 16 | 28 | 48 | 24 | 84 | 16 | 43 | 4 | 0 |
| mellitus, | (8.125%) | (2.827%) | (5.556%) | (9.877%) | (5.505%) | (18.584%) | (3.810%) | (26.543%) | (3.810%) | (0.000%) |
| No. (%) | | | | | | | | | | |
| Hypertension, | 3275 | 212 | 116 | 281 | 66 | 281 | 72 | 99 | 33 | 0 |
| No. (%) | (27.637%) | (37.456%) | (26.484%) | (57.819%) | (15.385%) | (62.168%) | (17.143%) | (61.111%) | (30.000%) | (0.000%) |
| BMI (kg/m²), | 26.818 | 26.367 | 25.557 | 27.500 | 27.349 | 27.462 | 26.945 | 27.698 | 26.022 | 26.330 |
| mean (SD) | (5.312) | (3.787) | (4.368) | (4.188) | (5.294) | (5.427) | (5.635) | (5.240) | (4.421) | (4.849) |
| HCV status, | 114 | 0 | 6 | 4 | 9 | 0 | 0 | 0 | 0 | 1 |
| No. (%) | (0.909%) | (0.000%) | (1.690%) | (0.825%) | (1.935%) | (0.000%) | (0.000%) | (0.000%) | (0.000%) | (6.250%) |
| Creatinine, | 1.071 | 0.936 | 0.967 | 1.153 | 1.048 | 0.873 | 0.786 | 0.867 | 0.760 | 1.672 |
| (mg/dL) | (0.737) | (0.400) | (0.531) | (0.523) | (0.809) | (0.405) | (0.303) | (0.302) | (0.340) | (1.852) |
| mean (SD) | | | | | | | | | | |
| Proteinuria, | 2798 | 123 | 139 | 53 | 165 | 52 | 3 | 15 | 52 | 0 |
| No. (%) | (30.125%) | (21.731%) | (44.127%) | (22.944%) | (38.642%) | (14.130%) | (3.614%) | (51.724%) | (44.828%) | (0.000%) |
| Number of | 34.120 | 54.272 | 16.580 | N/A | 26.127 | N/A | 38.463 | N/A | 27.731 | 23.783 |
| Glomeruli, | (29.780) | (32.991) | (5.102) | | (11.874) | | (18.285) | | (16.962) | (20.719) |
| mean (SD) | | | | | | | | | | |
| Arteriosclerosis | | | | | | | | | | |
| (cv) Banff | | | | | | | | | | |
| score, | | | | | | | | | | |
| No. (%) | | | | | | | | | | |
| 0 | 6478 | 410 | 281 | 203 | 425 | 195 | 89 | 110 | 120 | 55 |
| | (52.322%) | (72.438%) | (56.539%) | (41.856%) | (91.398%) | (42.952%) | (48.634%) | (60.440%) | (92.308%) | (61.111%) |
| 1 | 3684 | 145 | 142 | 268 | 25 | 223 | 63 | 55 | 6 | 30 |
| | (29.755%) | (25.618%) | (28.571%) | (55.258%) | (5.376%) | (49.119%) | (34.426%) | (30.220%) | (4.615%) | (33.333%) |
| 2 | 1987 | 7 | 55 | 13 | 13 | 35 | 20 | 15 | 3 | 4 |
| | (16.049%) | (1.237%) | (11.066%) | (2.680%) | (2.796%) | (7.709%) | (10.929%) | (8.242%) | (2.308%) | (4.444%) |
| 3 | 232 | 4 | 19 | 1 | 2 | 1 | 11 | 2 | 1 | 1 |
| | (1.874%) | (0.707%) | (3.823%) | (0.206%) | (0.430%) | (0.220%) | (6.011%) | (1.099%) | (0.769%) | (1.111%) |
| Arteriolar | | | | | | | | | | |
| hyalinosis | | | | | | | | | | |
| (ah) Banff | | | | | | | | | | |
| score, No. (%) | | | | | | | | | | |
| 0 | 7073 | 349 | 265 | 226 | 224 | 246 | 321 | 119 | 111 | 76 |
| | (61.574%) | (61.661%) | (51.859%) | (78.201%) | (48.172%) | (54.425%) | (78.870%) | (65.027%) | (86.719%) | (83.516%) |
| 1 | 3094 | 190 | 160 | 63 | 89 | 158 | 71 | 53 | 9 | 10 |
| | (26.935%) | (33.569%) | (31.311%) | (21.799%) | (19.140%) | (34.956%) | (17.445%) | (28.962%) | (7.031%) | (10.989%) |
| 2 | 1101 | 24 | 74 | 0 | 125 | 46 | 12 | 11 | 7 | 3 |
| | (9.585%) | (4.240%) | (14.481%) | (0.000%) | (26.882%) | (10.177%) | (2.948%) | (6.011%) | (5.469%) | (3.297%) |
| 3 | 219 | 3 | 12 | 0 | 27 | 2 | 3 | 0 | 1 | 2 |
| | (1.907%) | (0.530%) | (2.348%) | (0.000%) | (5.806%) | (0.442%) | (0.737%) | (0.000%) | (0.781%) | (2.198%) |
| Interstitial | | | | | | | | | | |
| fibrosis and | | | | | | | | | | |
| tubular atrophy | | | | | | | | | | |
| (IFTA) Banff | | | | | | | | | | |
| score, No. (%) | | | | | | | | | | |
| 0 | 7701 | 228 | 405 | 217 | 285 | 59 | 376 | 40 | 118 | 62 |
| | (60.188%) | (40.283%) | (77.586%) | (44.650%) | (61.290%) | (13.024%) | (88.679%) | (21.858%) | (90.769%) | (67.391%) |
| 1 | 4004 | 304 | 111 | 263 | 176 | 370 | 42 | 122 | 11 | 28 |
| | (31.293%) | (53.710%) | (21.264%) | (54.115%) | (37.849%) | (81.678%) | (9.906%) | (66.667%) | (8.462%) | (30.435%) |
| 2 | 1023 | 33 (5.830%) | 4 | 6 | 4 | 24 | 5 | 20 | 1 | 2 |
| | (7.995%) | | (0.766%) | (1.235%) | (0.860%) | (5.298%) | (1.179%) | (10.929%) | (0.769%) | (2.174%) |
| 3 | 67 | 1 (0.177%) | 2 | 0 | 0 | 0 | 1 | 1 | 0 | 0 |
| | (0.524%) | | (0.383%) | (0.000%) | (0.000%) | (0.000%) | (0.236%) | (0.546%) | (0.000%) | (0.000%) |
| Glomerulo- | 7.666 | 6.415 (8.988) | 9.947 | N/A | 7.029 | 7.643 | 7.732 | N/A | 6.220 | 3.891 |
| sclerosis, | (10.874) | | (11.671) | | (8.641) | (7.763) | (10.520) | | (13.101) | (5.863) |
| mean (SD) | | | | | | | | | | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.
*% was calculated among deceased donors.

TABLE 5
Baseline donor characteristics of the population cohort by donor type

| | | | | Deceased | | Living | |
|---|---|---|---|---|---|---|---|
| | | Overall | | Donor | | Donor | |
| | N | (n = 12,992) | N | (n = 9,449) | N | (n = 3,543) | p-value |
| Age (years), mean (SD) | 12992 | 49.8 (15.1) | 9449 | 51.7 (15.7) | 3543 | 44.5 (12.0) | <0.001 |
| Sex female, No. (%) | 12981 | 6082 (46.9%) | 9443 | 3979 (42.1%) | 3538 | 2103 (59.4%) | <0.001 |
| Donor type | | | | | | | |
| Deceased donor, No. (%) | 12992 | 9449 (72.7%) | 9449 | 9,449 (100%) | 3543 | 0 (0.0%) | <0.001 |
| Death from circulatory | 12947 | 1144 (8.8%) | 9405 | 1144 (12.2%) | 3542 | 0 (0.0%) | <0.001 |
| disease, No. | | | | | | | |
| Death from cerebrovascular | 12441 | 4936 (39.7%) | 9401 | 4936 (52.5%) | 3040 | 0 (0.0%) | <0.001 |
| disease, No. (%) | | | | | | | |
| Diabetes mellitus, No. (%) | 11003 | 894 (8.1%) | 7679 | 891 (11.6%) | 3324 | 3 (0.1%) | <0.001 |
| Hypertension, No. (%) | 11850 | 3275 (27.6%) | 8526 | 3260 (38.2%) | 3324 | 15 (0.5%) | <0.001 |
| BMI (kg/m²), mean (SD) | 11920 | 26.8 (5.3) | 8476 | 26.6 (5.5) | 3444 | 27.3 (4.9) | <0.001 |
| HCV status, No. (%) | 12540 | 114 (0.9%) | 9111 | 114 (1.3%) | 3429 | 0 (0.0%) | <0.001 |
| Creatinine (mg/dL), | 10912 | 1.1 (0.7) | 8516 | 1.1 (0.8) | 2396 | 0.9 (0.2) | <0.001 |
| mean (SD) | | | | | | | |
| Proteinuria, No. (%) | 9288 | 2798 (30.1%) | 6161 | 2798 (45.4%) | 3127 | 0 (0.0%) | <0.001 |
| Number of Glomeruli, | 7610 | 34.1 (29.8) | 6494 | 35.3 (31.3) | 1116 | 27.2 (17.0) | <0.001 |
| mean (SD) | | | | | | | |
| Arteriosclerosis (cv) | 12381 | | 9007 | | 3374 | | <0.001 |
| Banff score, No. (%) | | | | | | | |
| 0 | | 6478 (52.3%) | | 4513 (50.1%) | | 1965 (58.2%) | |
| 1 | | 3684 (29.8%) | | 2580 (28.6%) | | 1104 (32.7%) | |
| 2 | | 1987 (16.1%) | | 1699 (18.9%) | | 288 (8.5%) | |
| 3 | | 232 (1.9%) | | 215 (2.4%) | | 17 (0.5%) | |
| Arteriolar hyalinosis (ah) | 11487 | | 8038 | | 3449 | | <0.001 |
| Banff score, No. (%) | | | | | | | |
| 0 | | 7073 (61.6%) | | 4384 (54.5%) | | 2689 (78.0%) | |
| 1 | | 3094 (26.9%) | | 2459 (30.6%) | | 635 (18.4%) | |
| 2 | | 1101 (9.6%) | | 977 (12.2%) | | 124 (3.6%) | |
| 3 | | 219 (1.9%) | | 218 (2.7%) | | 1 (<0.1%) | |
| Interstitial fibrosis | 12795 | | 9338 | | 3457 | | <0.001 |
| and tubular atrophy | | | | | | | |
| (IFTA) Banff score, | | | | | | | |
| No. (%) | | | | | | | |
| 0 | | 7701 (60.2%) | | 5048 (54.1%) | | 2653 (76.7%) | |
| 1 | | 4004 (31.3%) | | 3220 (34.5%) | | 784 (22.7%) | |
| 2 | | 1023 (8.0%) | | 1006 (10.8%) | | 17 (0.5%) | |
| 3 | | 67 (0.5%) | | 64 (0.7%) | | 3 (0.1%) | |
| Glomerulosclerosis, | 7517 | 7.7 (10.9) | 6365 | 8.4 (11.3) | 1152 | 3.7 (7.1) | <0.001 |
| mean (SD) | | | | | | | |

TABLE 6
Summary of kidney day-zero histological lesion scores
(international Banff classification grading scheme)

| Banff lesion score | Abbreviation | Grading 0 | Grading 1 | Grading 2 | Grading 3 |
|---|---|---|---|---|---|
| Vascular fibrous intimal | CV | None | <=25% | 26-50% | >50% |
| thickening | | | | | |
| Arteriolar hyalinosis | ah | None | Mild to | Moderate | Severe in |
| | | | moderate | to severe | many |
| | | | in >=1 | in >1 | |
| Interstitial fibrosis | ci | <=5% | 6-25% | 26-50% | >50% |
| Tubular atrophy | ct | None | <=25% | 26-50% | >50% |

TABLE 7
Summary of participating centers' biopsy practices and procedures

| Country | Center | Performed | Timing | Technique | Tissue processing | Tissue stain | Interpretation |
|---|---|---|---|---|---|---|---|
| France | PTG | Surgeon | Preimplantation | Core needle | formalin-fixed | Periodic acid-Schiff (PAS), | Renal |
| | (Necker) | | (before anastomosis) | | paraffin-embedded | Masson's trichrome, | pathologist |
| | (SLS)(Toulouse) | | | | (FFPE) | Hematoxylin and eosin stain, | |
| | | | | | | Jones (methenamine silver) | |
| France | Kremlin Bicêtre | Surgeon | Procurement | Wedge, | frozen, | Periodic acid-Schiff (PAS), | General |
| | | | (organ retrieval), | Core needle | formalin-fixed | Masson's trichrome, | pathologist, |
| | | | Preimplantation | | paraffin-embedded | Hematoxylin and eosin stain, | Renal |
| | | | (before anastomosis) | | (FFPE) | Jones (methenamine silver) | pathologist |
| Belgium | UZ Leuven | Surgeon | Preimplantation | Core needle | frozen, | Periodic acid-Schiff (PAS), | Renal |
| | | | (before anastomosis) | | formalin-fixed | Masson's trichrome, | pathologist |
| | | | | | paraffin-embedded | Hematoxylin and eosin stain, | |
| | | | | | (FFPE) | Jones (methenamine silver) | |
| Belgium | CHU Liège | Surgeon | Postreperfusion | Wedge | formalin-fixed | Periodic acid-Schiff (PAS), | Renal |
| | | | (after the anastomosis) | | paraffin-embedded | Masson's trichrome, | pathologist |
| | | | | | (FFPE) | Hematoxylin and eosin stain | |
| Croatia | University hospital | Surgeon | Preimplantation | Wedge | formalin-fixed | Periodic acid-Schiff (PAS), | Renal |
| | centre Zagreb | | (before anastomosis) | | paraffin-embedded | Hematoxylin and eosin stain, | pathologist |
| | | | | | (FFPE) | Mallory-Weiss stain | |
| Spain | Hospital Clinic i | Surgeon | Procurement | Wedge | frozen | Hematoxylin and eosin stain | General |
| | Provincial | | (organ retrieval) | | | | pathologist |
| | de Barcelona | | | | | | |
| Spain | Vall d'Hebron | Surgeon | Procurement | Wedge | formalin-fixed | Hematoxylin and eosin stain | General |
| | University | | (organ retrieval) | | paraffin-embedded | | pathologist, |
| | Hospital | | | | (FFPE) | | Renal |
| | | | | | | | pathologist |
| Spain | Hospital | Surgeon | Procurement | Wedge, | frozen | Periodic acid-Schiff (PAS) | General |
| | Universitari de | | (organ retrieval) | Core needle | | | pathologist |
| | Bellvitge | | | | | | |
| Canada | University of | Surgeon | Postreperfusion | Wedge, | frozen, | Periodic acid-Schiff (PAS), | Renal |
| | Alberta | | (after the anastomosis) | Core needle | formalin-fixed | Masson's trichrome, | pathologist |
| | | | | | paraffin-embedded | Hematoxylin and eosin stain | |
| | | | | | (FFPE) | | |
| Canada | University of | Surgeon | Postreperfusion | Core needle | formalin-fixed | Periodic acid-Schiff (PAS), | Renal |
| | British Columbia | | (after the anastomosis) | | paraffin-embedded | Masson's trichrome, | pathologist |
| | | | | | (FFPE) | Hematoxylin and eosin stain | |
| United | Columbia University | Surgeon | Postreperfusion | Core needle | formalin-fixed | Periodic acid-Schiff (PAS), | Renal |
| States | Medical Center | | (after the anastomosis) | | paraffin-embedded | Masson's trichrome, | pathologist |
| | | | | | (FFPE) | Hematoxylin and eosin stain, | |
| | | | | | | Jones (methenamine silver) | |
| United | Mayo clinic | Surgeon | Postreperfusion | Core needle | formalin-fixed | Hematoxylin and eosin stain | Renal |
| States | | | (after the anastomosis) | | paraffin-embedded | | pathologist |
| | | | | | (FFPE) | | |
| United | UNOS | Surgeon | Procurement | Wedge, | Varies by OPO | Varies by OPO | Varies |
| States | | | (organ retrieval) | Core needle | | | by OPO |
| Australia | Royal Adelaide | Surgeon | Postreperfusion | Wedge | formalin-fixed | Periodic acid-Schiff (PAS), | Renal |
| | Hospital | | (after the anastomosis) | | paraffin-embedded | Hematoxylin and eosin stain | pathologist |
| | | | | | (FFPE) | | |

TABLE 8
Training and test sets baseline characteristics
Table 8.1 : Arteriosclerosis (cv) day-zero histological lesion

| | Overall | | Training set | | Test set | | |
|---|---|---|---|---|---|---|---|
| | (n = 12,992) | | (n = 9,746) | | (n = 3,246) | | p-value |
| Age (years), mean (SD) | 49.751 | (15.134) | 49.794 | (15.100) | 49.620 | (15.237) | 0.573 |
| Sex female, No. (%) | 6086 | (46.844%) | 4579 | (46.983%) | 1507 | (46.426%) | 0.596 |
| Donor type, No. (%) | | | | | | | |
| Deceased donor, No. (%) | 9449 | (72.729%) | 7054 | (72.378%) | 2395 | (73.783%) | 0.125 |
| Death from circulatory disease, | 1144 | (8.805%) | 859 | (8.814%) | 285 | (8.780%) | 0.982 |
| No. (%) | | | | | | | |
| Death from cerebrovascular | 4947 | (38.077%) | 3684 | (37.800%) | 1263 | (38.909%) | 0.369 |
| disease, No. (%) | | | | | | | |
| Diabetes mellitus, No. (%) | 957 | (7.366%) | 738 | (7.572%) | 219 | (6.747%) | 0.328 |
| Hypertension, No. (%) | 3439 | (26.470%) | 2579 | (26.462%) | 860 | (26.494%) | 0.99 |
| BMI (kg/m²), mean (SD) | 26.755 | (5.121) | 26.736 | (5.157) | 26.811 | (5.012) | 0.462 |
| HCV status, No. (%) | 115 | (0.885%) | 90 | (0.923%) | 25 | (0.770%) | 0.484 |
| Creatinine (mg/dL), mean (SD) | 1.058 | (0.680) | 1.059 | (0.690) | 1.053 | (0.649) | 0.653 |
| Proteinuria, No. (%) | 4880 | (37.562%) | 3646 | (37.410%) | 1234 | (38.016%) | 0.551 |
| cv, No. (%) | | | | | | | 0.937 |
| 0 | 6851 | (52.732%) | 5135 | (52.688%) | 1716 | (52.865%) | |
| 1 | 3861 | (29.718%) | 2892 | (29.674%) | 969 | (29.852%) | |
| 2 | 2045 | (15.740%) | 1539 | (15.791%) | 506 | (15.588%) | |
| 3 | 235 | (1.809%) | 180 | (1.847%) | 55 | (1.694%) | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.

TABLE 8.2
Arteriolar hyalinosis (ah) day-zero histological lesion

| | Overall | | Training set | | Test set | | |
|---|---|---|---|---|---|---|---|
| | (n = 12,992) | | (n = 9,746) | | (n = 3,246) | | p-value |
| Age (years), mean (SD) | 49.751 | (15.134) | 49.810 | (15.126) | 49.574 | (15.158) | 0.443 |
| Sex female, No. (%) | 6086 | (46.844%) | 4568 | (46.871%) | 1518 | (46.765%) | 0.933 |
| Donor type, No. (%) | | | | | | | |
| Deceased donor, No. (%) | 9449 | (72.729%) | 7082 | (72.666%) | 2367 | (72.921%) | 0.795 |
| Death from circulatory disease, | 1144 | (8.805%) | 862 | (8.845%) | 282 | (8.688%) | 0.812 |
| No. (%) | | | | | | | |
| Death from cerebrovascular disease, | 4947 | (38.077%) | 3697 | (37.934%) | 1250 | (38.509%) | 0.573 |
| No. (%) | | | | | | | |
| Diabetes mellitus, No. (%) | 957 | (7.366%) | 706 | (7.244%) | 251 | (7.733%) | 0.477 |
| Hypertension, No. (%) | 3439 | (26.470%) | 2554 | (26.206%) | 885 | (27.264%) | 0.446 |
| BMI (kg/m²), mean (SD) | 26.755 | (5.121) | 26.776 | (5.149) | 26.691 | (5.036) | 0.408 |
| HCV status, No. (%) | 115 | (0.885%) | 91 | (0.934%) | 24 | (0.739%) | 0.360 |
| Creatinine (mg/dL), mean (SD) | 1.058 | (0.680) | 1.056 | (0.682) | 1.065 | (0.674) | 0.491 |
| Proteinuria, No. (%) | 4880 | (37.562%) | 3648 | (37.431%) | 1232 | (37.954%) | 0.608 |
| ah, No. (%) | | | | | | | |
| 0 | 7319 | (56.335%) | 5493 | (56.362%) | 1826 | (56.254%) | 0.999 |
| 1 | 3522 | (27.109%) | 2640 | (27.088%) | 882 | (27.172%) | |
| 2 | 1568 | (12.069%) | 1175 | (12.056%) | 393 | (12.107%) | |
| 3 | 583 | (4.487%) | 438 | (4.494%) | 145 | (4.467%) | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.

TABLE 8.3
Interstitial fibrosis and tubular atrophy (IFTA) day-zero histological lesion

| | Overall | | Training set | | Test set | | |
|---|---|---|---|---|---|---|---|
| | (n = 12,992) | | (n = 9,745) | | (n = 3,247) | | p-value |
| Age (years), mean (SD) | 49.751 | (15.134) | 49.748 | (15.095) | 49.759 | (15.253) | 0.971 |
| Sex female, No. (%) | 6086 | (46.844%) | 4583 | (47.029%) | 1503 | (46.289%) | 0.477 |
| Donor type, No. (%) | | | | | | | |
| Deceased donor, No. (%) | 9449 | (72.729%) | 7064 | (72.488%) | 2385 | (73.452%) | 0.496 |
| Death from circulatory disease, | 1144 | (8.805%) | 842 | (8.640%) | 302 | (9.301%) | 0.465 |
| No. (%) | | | | | | | |
| Death from cerebrovascular disease, | 4947 | (38.077%) | 3711 | (38.081%) | 1236 | (38.066%) | 1.000 |
| No. (%) | | | | | | | |
| Diabetes mellitus, No. (%) | 957 | (7.366%) | 725 | (7.440%) | 232 | (7.145%) | 0.605 |
| Hypertension, No. (%) | 3439 | (26.470%) | 2618 | (26.865%) | 821 | (25.285%) | 0.181 |
| BMI (kg/m²), mean (SD) | 26.755 | (5.121) | 26.812 | (5.148) | 26.584 | (5.037) | 0.027 |
| HCV status, No. (%) | 115 | (0.885%) | 80 | (0.821%) | 35 | (1.078%) | 0.213 |
| Creatinine (mg/dL), mean (SD) | 1.058 | (0.680) | 1.064 | (0.689) | 1.040 | (0.652) | 0.076 |
| Proteinuria, No. (%) | 4880 | (37.562%) | 3650 | (37.455%) | 1230 | (37.881%) | 0.679 |
| IFTA, No. (%) | | | | | | | 0.997 |
| 0 | 7838 | (60.329%) | 5880 | (60.339%) | 1958 | (60.302%) | |
| 1 | 4058 | (31.235%) | 3042 | (31.216%) | 1016 | (31.290%) | |
| 2 | 1029 | (7.920%) | 772 | (7.922%) | 257 | (7.915%) | |
| 3 | 67 | (0.516%) | 51 | (0.523%) | 16 | (0.493%) | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.

TABLE 8.4
Glomerulosclerosis day-zero histological lesion

| | Overall | | Training set | | Test set | | |
|---|---|---|---|---|---|---|---|
| | (n = 12,992) | | (n = 9,746) | | (n = 3,246) | | p-value |
| Age (years), mean (SD) | 49.751 | (15.134) | 49.739 | (15.147) | 49.787 | (15.096) | 0.874 |
| Sex female, No. (%) | 6086 | (46.844%) | 4566 | (46.850%) | 1520 | (46.827%) | 0.998 |
| Donor type, No. (%) | | | | | | | |
| Deceased donor, No. (%) | 9449 | (72.729%) | 7043 | (72.266%) | 2406 | (74.122%) | 0.042 |
| Death from circulatory disease, | 1144 | (8.805%) | 849 | (8.711%) | 295 | (9.088%) | 0.535 |
| No. (%) | | | | | | | |
| Death from cerebrovascular disease, | 4947 | (38.077%) | 3700 | (37.964%) | 1247 | (38.417%) | 0.661 |
| No. (%) | | | | | | | |
| Diabetes mellitus, No. (%) | 957 | (7.366%) | 717 | (7.357%) | 240 | (7.394%) | 0.975 |
| Hypertension, No. (%) | 3439 | (26.470%) | 2576 | (26.431%) | 863 | (26.587%) | 0.880 |
| BMI (kg/m²), mean (SD) | 26.755 | (5.121) | 26.782 | (5.172) | 26.671 | (4.967) | 0.275 |
| HCV status, No. (%) | 115 | (0.885%) | 87 | (0.893%) | 28 | (0.863%) | 0.960 |
| Creatinine (mg/dL), mean (SD) | 1.058 | (0.680) | 1.053 | (0.666) | 1.074 | (0.720) | 0.127 |
| Proteinuria, No. (%) | 4880 | (37.562%) | 3619 | (37.133%) | 1261 | (38.848%) | 0.084 |
| Glomerulosclerosis, mean (SD) | 7.824 | (9.555) | 7.796 | (9.463) | 7.906 | (9.828) | 0.578 |

Abbreviations: BMI: body mass index. HCV: hepatitis C virus.
Proteinuria values were positive when dipstick greater than or equal to 1 or urine protein to creatinine ratio (UPCR, g/g) greater
than or equal to 0.5 g/g.

TABLE 9
Baseline donor characteristics of the population cohort before and after
imputation comparison

| | | Before | | | After | | |
|---|---|---|---|---|---|---|---|
| | | Imputation | | | Imputation | | |
| | N | (n = 12,992) | | N | (n = 12,992) | | p-value |
| Age (years), | 12992 | 49.751 | (15.134) | 12992 | 49.751 | (15.134) | 1.000 |
| mean (SD) | | | | | | | |
| Sex female, | 12981 | 6082 | (46.853%) | 12992 | 6086 | (46.844%) | 0.998 |
| No. (%) | | | | | | | |
| Donor type, No. (%) | | | | | | | |
| Deceased donor, | 12992 | 9449 | (72.729%) | 12992 | 9449 | (72.729%) | 1.000 |
| No. (%) | | | | | | | |
| Death from circulatory disease, | 12947 | 1144 | (8.836%) | 12992 | 1144 | (8.805%) | 0.948 |
| No. (%) | | | | | | | |
| Death from cerebrovascular | 12441 | 4936 | (39.675%) | 12992 | 4947 | (38.077%) | 0.009 |
| disease, | | | | | | | |
| No. (%) | | | | | | | |
| Diabetes mellitus, No. (%) | 11003 | 894 | (8.125%) | 12992 | 957 | (7.366%) | 0.030 |
| Hypertension, No. (%) | 11850 | 3275 | (27.637%) | 12992 | 3439 | (26.470%) | 0.040 |
| BMI (kg/m²), mean (SD) | 11920 | 26.818 | (5.312) | 12992 | 26.755 | (5.121) | 0.341 |
| HCV status, No. (%) | 12540 | 114 | (0.909%) | 12992 | 115 | (0.885%) | 0.892 |
| Creatinine (mg/dL), | 10912 | 1.071 | (0.737) | 12992 | 1.058 | (0.680) | 0.150 |
| mean (SD) | | | | | | | |
| Proteinuria, No. (%) | 9288 | 2798 | (30.125%) | 12992 | 4880 | (37.562%) | <0.001 |
| Number of Glomeruli, | 7610 | 34.120 | (29.780) | 7610 | 34.120 | (29.780) | 1.000 |
| mean (SD) | | | | | | | |
| Arteriosclerosis (cv) | 12381 | | | 12992 | | | 0.866 |
| Banff score, No. (%) | | | | | | | |
| 0 | | 6478 | (52.322%) | | 6851 | (52.732%) | |
| 1 | | 3684 | (29.755%) | | 3861 | (29.718%) | |
| 2 | | 1987 | (16.049%) | | 2045 | (15.740%) | |
| 3 | | 232 | (1.874%) | | 235 | (1.809%) | |
| Arteriolar hyalinosis (ah) | 11487 | | | 12992 | | | <0.001 |
| Banff score, No. (%) | | | | | | | |
| 0 | | 7073 | (61.574%) | | 7319 | (56.335%) | |
| 1 | | 3094 | (26.935%) | | 3522 | (27.109%) | |
| 2 | | 1101 | (9.585%) | | 1568 | (12.069%) | |
| 3 | | 219 | (1.907%) | | 583 | (4.487%) | |
| Interstitial fibrosis and tubular | 12795 | | | 12992 | | | 0.994 |
| atrophy (IFTA) Banff score, | | | | | | | |
| No. (%) | | | | | | | |
| 0 | | 7701 | (60.188%) | | 7838 | (60.329%) | |
| 1 | | 4004 | (31.293%) | | 4058 | (31.235%) | |
| 2 | | 1023 | (7.995%) | | 1029 | (7.920%) | |
| 3 | | 67 | (0.524%) | | 67 | (0.516%) | |
| Glomerulosclerosis, | 7517 | 7.666 | (10.874) | 12992 | 7.824 | (9.555) | 0.296 |
| mean (SD) | | | | | | | |

Abbreviations: BMI: body mass index. HCV: hepatitis C virus.
Proteinuria values were positive when dipstick greater than or equal to 1 or urine protein to creatinine ratio (UPCR, g/g) greater than or equal to 0.5 g/g.

Table 10: Calibration Confusion Matrices of the Virtual Biopsy System

Model calibration performances were measured with confusion matrices since day-zero lesion scores comprise multiclass scores.

TABLE 10.1
Confusion matrix on the arteriosclerosis (cv biopsy lesion score) test set

| Reference | Virtual Biopsy Prediction | | | |
|---|---|---|---|---|
| (test set) | 0 | 1 | 2 | 3 |
| 0 | 1324 | 238 | 136 | 18 |
| 1 | 413 | 389 | 150 | 17 |
| 2 | 130 | 116 | 244 | 16 |
| 3 | 9 | 15 | 22 | 9 |

TABLE 10.2
Confusion matrix on the arteriolar hyalinosis (ah biopsy lesion score)
test set

| Reference | Virtual Biopsy Prediction | | | |
|---|---|---|---|---|
| (test set) | 0 | 1 | 2 | 3 |
| 0 | 1479 | 243 | 80 | 24 |
| 1 | 390 | 375 | 81 | 36 |
| 2 | 102 | 85 | 169 | 37 |
| 3 | 11 | 19 | 18 | 97 |

TABLE 10.3
Confusion matrix on the interstitial fibrosis and tubular atrophy
(IFTA biopsy lesion score) test set

| Reference | Virtual Biopsy Prediction | | | |
|---|---|---|---|---|
| (test set) | 0 | 1 | 2 | 3 |
| 0 | 1570 | 319 | 57 | 12 |
| 1 | 410 | 536 | 60 | 10 |
| 2 | 28 | 82 | 143 | 4 |
| 3 | 1 | 8 | 3 | 4 |

TABLE 11
Validation of the virtual biopsy system in different subpopulations and clinical scenarios

| | | Discrimination | | | |
|---|---|---|---|---|---|
| | | | | Interstitial | |
| | | | | fibrosis | |
| | | | Arteriolar | and tubular | |
| Scenarios and | | Arteriosclerosis | hyalinosis | atrophy | |
| subpopulations | | (cv)* | (ah)* | (IFTA)* | Glomerulosclerosis† |
| Continent | Europe | 0.690 | 0.710 | 0.701 | 6.6 |
| | North America | 0.777 | 0.847 | 0.820 | 3.056 |
| | Australia | 0.775 | 0.790 | 0.755 | 5.643 |
| Ethnicity‡ | African | 0.806 | 0.934 | 0.868 | 5.054 |
| | American | | | | |
| | Non-African | 0.770 | 0.842 | 0.811 | 4.086 |
| | American | | | | |
| Type of | Preimplantation | 0.690 | 0.712 | 0.700 | 6.623 |
| biopsy | (before | | | | |
| | anastomosis) | | | | |
| | Postreperfusion | 0.735 | 0.750 | 0.747 | 2.966 |
| | (after | | | | |
| | anastomosis) | | | | |

*multi-AUC was used to measure the performance.
†MAE was used to measure the performance.
‡Missing ethnicity values were removed.
Abbreviation: area under the curve (AUC, higher the better), mean absolute error (MAE, lower the better).
All subpopulations were stratified from test sets.

1. A method for assessing at least one histological piece of information of an organ of a subject, the method being computer-implemented and the method comprising:
providing parameters relative to the subject, to obtain provided parameters, and
for each of the at least one histological piece of information, applying a predicting function on the provided parameters to obtain an assessed histological piece of information,
the assessed histological piece of information being a numerical value for the organ when the histological piece of information is a numerical value or the assessed histological piece of information being probabilities of belonging to different predefined classes for the organ when the histological piece of information is a belonging to a predefined class among the different predefined classes, and
each predicting function being specific to the considered histological piece of information and being obtained by using an artificial intelligence technique.

2. The method for assessing at least one histological piece of information according to claim 1, wherein, for each of the at least one histological piece of information, the artificial intelligence technique comprises:
a phase of preparing a data set formed by elements, each element associating to subject parameters the assessed histological piece of information,
a phase of training a plurality of models, to obtain trained models, and
a phase of obtaining the predicting function comprising:
selecting models among the plurality of trained models based on a performance criteria, to obtain selected models, and
obtaining the predicting function as a aggregating function of the selected models.

3. The method for assessing at least one histological piece of information according to claim 1, wherein:
the organ is a kidney, the histological pieces of information being the value of the glomerusclerosis and the predefined class being the stages of the arteriosclerosis, the stages of the arteriolar hyalinosis and the stages of the interstitial fibrosis/tubular atrophy.

4. The method for assessing at least one histological piece of information according to claim 1, wherein the organ is a graft donor, the provided subject parameters comprising at least one piece of information chosen among the list consisting of:
the comorbidities,
a clinical data, and
a biological data.

5. The method for assessing at least one histological piece of information according to claim 2, wherein the phase of preparing a data set formed by elements comprises carrying out at least one preparation procedure, the preparation procedure being a preparation technique chosen among:
a first procedure comprising collecting initial elements, and completing the initial elements by using an imputation technique, the imputation technique comprising using a random forest technique,
a second procedure comprising splitting the data set into a training set and a testing set, and
a third procedure comprising the phase of preparing comprises a standardization of the subject parameters.

6. The method for assessing at least one histological piece of information according to claim 2, wherein, when the histological piece of information is a belonging to a predefined class among different predefined classes and the different predefined classes being superior or equal to 4, the initial training data set comprises a respective number of elements for each predefined class of the considered histological piece of information, the phase of preparing comprising itering an operation of replacing randomly an element present in the training data set with a first number superior to at least one other numbers by elements present in the training data set with an inferior number to the first number until the number of elements for each predefined class be the same in the obtained training data set.

7. The method for assessing at least one histological piece of information according to claim 6, wherein the phase of training comprises penalizing in case of mispredicting of the two uppest classes and/or, wherein each model comprises at least one hyperparameter for controlling the training process and the phase of training comprising hyperparameter tuning.

8. The method for assessing at least one histological piece of information according to claim 2, wherein the phase of training comprises creating heterogeneities in the set of data.

9. The method for assessing at least one histological piece of information according to claim 2, wherein the models are chosen in the list consisting of:
a linear model,
a non-linear model,
an ensemble model, and
a deep learning model.

10. The method for assessing at least one histological piece of information according to claim 1, wherein the artificial technique comprising an evaluation phase, the evaluation phase comprises carrying out at least one evaluation procedure, the evaluation procedure being an evaluation procedure chosen among:
a first procedure comprising applying multi-AUC of unweighted pairwise discriminability of classes when the histological piece of information is a belonging to a predefined class among the different predefined classes,
a second procedure comprising, for each histological piece of information which is a numerical value, calculating the mean absolute error between the predicted value and the measured value for the histological piece of information,
a third technique comprising using a robustness test and/or a durability test,
a fourth technique comprising a random forest algorithm, and
a fifth technique comprising using a bootstrapping technique.

11. The method for assessing at least one histological piece of information according to claim 2, wherein the aggregating function is chosen in the list consisting of: simple average, weighted average, majority voting, weighted voting and ensemble stacking.

12. Method selected from the group consisting of:
a method for predicting that a subject is at risk of suffering from a disease, the method for predicting comprising at least the steps of:
carrying out the steps of a method for assessing at least one histological piece of information according to claim 1 wherein the step of providing is achieved by receiving the parameters relative to the subject at risk of suffering from a disease, to obtain assessed histological pieces of information, and
predicting that the subject is at risk of suffering from the disease based on the assessed histological pieces of information,
a method for diagnosing a disease to a subject, the method for diagnosing comprising at least the steps of:
carrying out the steps of the method for assessing at least one histological piece of information according to claim 1 wherein the step of providing is achieved by receiving the parameters relative to the subject, to obtain assessed histological pieces of information, and
diagnosing the disease based on the assessed histological pieces of information,
a method for identifying a therapeutic target for preventing and/or treating a disease, the method comprising at least the steps of:
carrying out the steps of the method for assessing at least one histological piece of information of an organ of a first subject, to obtain first assessed histological pieces of information, wherein the first subject is suffering from the disease and the method for assessing is according to claim 1 wherein the step of providing is achieved by receiving the parameters relative to the subject,
carrying out the steps of the method for assessing at least one histological piece of information of an organ of a second subject, to obtain second assessed histological pieces of information, wherein the second subject is not suffering from the disease and the method for assessing is according to claim 1 wherein the step of providing is achieved by receiving the parameters relative to the subject, and
selecting a therapeutic target based on the comparison of the first and second assessed histological pieces of information,
a method for identifying a biomarker for a disease, the biomarker being a diagnosis biomarker of the disease, a susceptibility biomarker of the disease, a prognostic biomarker of the disease or a predictive biomarker in response to the treatment of the disease, the method comprising at least the steps of:
carrying out the steps of the method for assessing at least one histological piece of information of an organ of a first subject, to obtain first assessed histological pieces of information, wherein the first subject is suffering from the disease and the method for assessing is according to claim 1 wherein the step of providing is achieved by receiving the parameters relative to the subject,
carrying out the steps of the method for assessing at least one histological piece of information of an organ of a second subject, to obtain second assessed histological pieces of information, wherein the second subject is not suffering from the disease and the method for assessing is according to claim 1 wherein the step of providing is achieved by receiving the parameters relative to the subject, and
selecting a biomarker target based on the comparison of the first and second assessed histological pieces of information, and
a method for screening a compound useful as a medicament, the compound having an effect on a known therapeutical target for preventing and/or treating a disease, the method comprising at least the steps of:
carrying out the steps of the method for assessing at least one histological piece of information of an organ of a first subject, to obtain first assessed histological pieces of information, wherein the first subject is from the disease and has received the compound and the method for assessing is according to claim 1 wherein the step of providing is achieved by receiving the parameters relative to the subject,
carrying out the steps of the method for assessing at least one histological piece of information of an organ of a second subject, to obtain second assessed histological pieces of information, wherein the second subject is suffering from the disease and has not received the compound and the method for assessing is according to claim 1 wherein the step of providing is achieved by receiving the parameters relative to the subject, and
selecting a biomarker target based on the comparison of the first and second assessed histological pieces of information, and
a method for monitoring patients enrolled in a clinical trial to provide a quantitative measure for the therapeutic efficacy of the therapy which is subject to the clinical trial by carrying out the steps of the method for assessing at least one histological piece of information of an organ of said patients, the method for assessing being according to claim 1 wherein the step of providing is achieved by receiving the parameters relative to the subject.

13. Computer program product comprising computer program instructions, the computer program instructions being loadable into a data-processing unit and adapted to cause execution of a method according to claim 1 when run by the data-processing unit.

14. Computer-readable medium comprising computer program instructions which, when executed by a data-processing unit, cause execution of a method according to claim 1.

15. The method for assessing at least one histological piece of information according to claim 1, wherein the organ is a heart, the histological pieces of information being the stages of the acute cellular rejection, or the stages of the antibody-mediated rejection.

16. The method for assessing at least one histological piece of information according to claim 1, wherein the organ is a lung, the histological pieces of information being the stages of the acute cellular rejection, or the stages of the antibody-mediated rejection.
